(19) United States
(12) Patent Application Publication
(10) Pub. No.: US 2004/0092716 A1
(43) Pub. Date: May 13, 2004

(54) Steroidogenic factor-1 protein variants and methods of making same

(76) Inventors: Holly A. Ingraham, San Francisco, CA (US); Irina Krylova, Oakland, CA (US)

Correspondence Address: Cathryn Campbell, McDERMOTT, WILL & EMERY, 7th Floor, 4370 La Jolla Village Drive, San Diego, CA, 92122, US

(21) Appl. No.: 10/616,897

(22) Filed: Jul. 9, 2003

Related U.S. Application Data
(60) Provisional application No. 60/395,371, filed on Jul. 12, 2002.

(51) Int. Cl. C07K 14/72; C07H 21/04

(52) U.S. Cl. 530/350; 536/23.5; 435/69.1; 435/320.1; 435/325

(57) ABSTRACT

The present invention provides a properly folded steroidogenic factor-1 (SF-1)-like receptor variant, or active fragment thereof, which has an amino acid sequence that encodes a SF-1-like receptor or an active fragment thereof and that lacks at least one naturally occurring cysteine residue within the ligand-binding domain of the receptor. Such a properly folded SF-1 receptor variant or active fragment thereof can exhibit increased monomer stability as compared to analogous receptor retaining the cysteine residues lacking in the variant. In one embodiment, at least one naturally occurring cysteine residue within the ligand-binding domain of the SF-1-like receptor is substituted with a non-cysteine residue such as serine or threonine.

This application claims benefit of the filing date of U.S. Provisional Application No. 60/395,371, filed Jul. 12, 2002, and which is incorporated herein by reference.

This invention was made with government support under P01 DK58390 from the National Institute of Health. The government has certain rights in this invention.

### BACKGROUND OF THE INVENTION

1. Field of the Invention

The present invention relates generally to the fields of nuclear receptors and protein expression and, more specifically, to mutants of steroidogenic factor-1 and related monomeric nuclear receptors with improved properties.

2. Background Information

The orphan nuclear receptor steroidogenic factor-1 (SF-1) is a monomeric binding nuclear receptor that is essential for endocrine organ proliferation, peptide hormone expression and for all steroid metabolism. Recombinant SF-1 protein has been expressed; in E. coli, SF-1 protein is soluble and expressed at reasonable levels. However, a variety of in vitro assays require high concentrations of purified protein. Unfortunately, SF-1 aggregates and is unsuitable for use at high concentrations in vitro. As an example, relatively high concentrations of conformationally uniform protein are required to prepare crystals suitable for X-ray crystallography studies. Because wild type protein produces twinned crystals, X-ray structural analysis cannot be performed. Similarly, wild type protein is not monomeric at the concentrations needed for functional analysis of the receptor using in vitro peptide binding assays. In brief, any application that requires high levels of purified protein cannot be performed with the SF-1 protein preparations currently available. Thus, there is a need for novel forms of SF-1 that exhibit increased stability of the monomeric form of the receptor and that remain monomeric at high protein concentrations. The present invention satisfied this need and provides related advantages as well.

### SUMMARY OF THE INVENTION

The present invention provides a properly folded steroidogenic factor-1 (SF-1)-like receptor variant, or active fragment thereof, which has an amino acid sequence that encodes a SF-1-like receptor or an active fragment thereof and that lacks at least one naturally occurring cysteine residue within the ligand-binding domain of the receptor. Such a properly folded SF-1 receptor variant or active fragment thereof can exhibit increased monomer stability as compared to the analogous receptor that retains the cysteine residues lacking in the variant. In one embodiment, at least one naturally occurring cysteine residue within the ligand-binding domain of the SF-1-like receptor is substituted with a non-cysteine residue.

A SF-1-like receptor variant or active fragment of the invention can be, for example, a SF-1 receptor variant or a liver related homolog 1/fetoprotein transcription factor (LRH-1/FTF/SF-1β) receptor variant, or an active fragment thereof. An active fragment of the invention can include, for example, the ligand-binding domain of a SF-1-like receptor. In one embodiment, the invention provides an active fragment containing a SF-1 like receptor variant ligand-binding domain without additional sequence from the same receptor. In another embodiment, the invention provides an active fragment containing a ligand-binding domain and activation function 1 (AF1) of a SF-1-like receptor. In a further embodiment, the active fragment contains a ligand-binding domain and AF1 without additional sequence from the same receptor.

A SF-1-like receptor variant or active fragment of the invention lacks at least one naturally occurring cysteine residue within the ligand-binding domain of the receptor. In one embodiment, the invention provides a SF-1-like receptor variant, or active fragment thereof, which lacks at least two naturally occurring cysteine residues within the ligand-binding domain of the receptor. In another embodiment, the invention provides a SF-1-like receptor variant or active fragment thereof, in which at least two naturally occurring cysteine residues within the ligand-binding domain are substituted with non-cysteine residues. In a further embodiment, the invention provides a SF-1-like receptor variant, or active fragment thereof, which lacks at least three naturally occurring cysteine residues within the ligand-binding domain of the receptor. If desired, at least three naturally occurring cysteine residues within the ligand-binding domain of the SF-1-like receptor variant can be substituted with non-cysteine residues.

A variety of amino acid substitutions can be useful in the SF-1-like receptor variants and active fragments of the invention. In particular embodiments, the ligand-binding domain of a SF-1-like receptor or active fragment thereof contains substitutions of at least one, two or three naturally occurring cysteine residues, where each of the cysteine residues is independently substituted with serine, threonine, alanine, valine, glycine, leucine, isoleucine, tyrosine, phenylalanine, tryptophan, methionine or histidine. In further embodiments, at least one, two or three naturally occurring cysteine residues each is independently substituted with serine or threonine. In still further embodiments, at least one, two or three naturally occurring cysteine residues each is independently substituted with serine.

Further provided by the invention is a SF-1-like receptor variant, such as a SF-1 or LRH-1 receptor variant, or an active fragment thereof, which contains amino acid substitutions at cysteine residues corresponding to C267, C302 and C423 of murine SF-1. In one embodiment, the invention provides a SF-1 receptor variant, or active fragment thereof, containing amino acid substitutions at cysteine residues corresponding to C267, C302 and C423 of murine SF-1. Such a SF-1 receptor variant can be, for example, a human SF-1 receptor variant. In another embodiment, the invention provides a SF-1 receptor variant, or active fragment thereof, containing serine substitutions at cysteine residues corresponding to C267, C302 and C423 of murine SF-1. In a further embodiment, the invention provides a human SF-1 receptor variant, or active fragment thereof, containing serine substitutions at cysteine residues corresponding to C267, C302 and C423 of murine SF-1.

The invention also provides a SF-1-like receptor variant, such as a SF-1 or LRH-1 receptor variant, or an active fragment thereof, which contains amino acid substitutions at cysteine residues corresponding to C302 and C423 of murine SF-1. Such a SF-1 receptor variant, or active fragment thereof, can contain, for example, amino acid substitutions at cysteine residues corresponding to C302 and C423 of murine SF-1. In one embodiment, the invention provides a human SF-1 receptor variant, or active fragment thereof, containing amino acid substitutions at cysteine residues corresponding to C302 and C423 of murine SF-1. In another embodiment, the invention provides a SF-1 receptor variant, or active fragment thereof, that contains serine substitutions at cysteine residues corresponding to C302 and C423 of murine SF-1. In yet a further embodiment, the invention provides a human SF-1 receptor variant, or active fragment thereof, that contains serine substitutions at cysteine residues corresponding to C302 and C423 of murine SF-1.

The present invention additionally provides a SF-1-like receptor variant, such as a SF-1 or LRH-1 receptor variant, or an active fragment thereof, which contains amino acid substitutions at cysteine residues corresponding to C408 and C413 of murine SF-1. In one embodiment, the invention provides a SF-1 receptor variant, or active fragment thereof, containing amino acid substitutions at cysteine residues corresponding to C408 and C413 of murine SF-1. In another embodiment, the invention provides a human SF-1 receptor variant, or active fragment thereof, containing amino acid substitutions at cysteine residues corresponding to C408 and C413 of murine SF-1. In a further embodiment, the invention provides a SF-1 receptor variant, or active fragment thereof, containing serine substitutions at cysteine residues corresponding to C408 and C413 of murine SF-1. As a non-limiting example, such a SF-1 receptor variant can be a human SF-1 receptor variant.

Further provided herein is an LRH-1 receptor variant having amino acid substitutions at cysteine residues corresponding to C267 and C302 of murine SF-1. Such an LRH-1 receptor variant of can have, for example, serine substitutions at cysteine residues corresponding to C267 and C302 of murine SF-1, and further can be, for example, a human LRH-1 receptor variant. The invention also provides an LRH-1 receptor variant having an amino acid substitution at a cysteine residue corresponding to C302 of murine SF-1. Such a substitution can be, without limitation, a serine substitution, and such an LRH-1 receptor variant can be, for example, a human LRH-1 receptor variant. Also provided herein is an LRH-1 receptor variant having an amino acid substitution at a cysteine residue corresponding to C408 of murine SF-1. Amino acid substitutions useful in such an LRH-1 receptor variant include, but are not limited to, serine substitutions. A variety of LRH-1 receptor variants can have an amino acid substitution at a cysteine residue corresponding to C408 of murine SF-1 including, for example, a human LRH-1 receptor variant.

The present invention further provides a properly folded SF-1 receptor variant, or active fragment thereof, which has an amino acid sequence encoding a SF-1 receptor or an active fragment thereof and that lacks at least one of the following naturally occurring cysteine residues: a cysteine residue corresponding to C408 of murine SF-1 or a cysteine residue corresponding to C413 of murine SF-1. Such a SF-1 receptor variant or active fragment of the invention can exhibit increased monomer stability as compared to an analogous receptor that retains all cysteine residues lacking in the variant. In one embodiment, the invention provides a SF-1 receptor variant or active fragment in which a cysteine residue corresponding to C408 or C413 of murine SF-1 is substituted with a non-cysteine residue. In another embodiment, the invention provides a SF-1 receptor variant or active fragment having an amino acid sequence that lacks naturally occurring cysteine residues corresponding to C408 and C413 of murine SF-1. Such a SF-1 receptor variant or active fragment can have, for example, amino acid substitutions at cysteine residues corresponding to C408 and C413 of murine SF-1. In a further embodiment, cysteine residues corresponding to C408 and C413 of murine SF-1 are independently substituted with serine, threonine, alanine or valine. In yet a further embodiment, cysteine residues corresponding to C408 and C413 of murine SF-1 are independently substituted with serine or threonine. In still a further embodiment, cysteine residues corresponding to C408 and C413 of murine SF-1 are substituted with serine. Any of the above SF-1 receptor active fragments can be, for example, an active fragment that includes a ligand-binding domain and further can be, for example, an active fragment that includes a ligand-binding domain without additional SF-1 receptor sequence.

In another embodiment, the invention provides a human SF-1 receptor variant or active fragment thereof which has an amino acid sequence encoding a human SF-1 receptor or an active fragment thereof and that lacks a cysteine residue corresponding to C408 or C413 of murine SF-1. In a further embodiment, the invention provides a murine SF-1 receptor variant or active fragment thereof which has an amino acid sequence encoding a murine SF-1 receptor or an active fragment thereof and that lacks a cysteine residue corresponding to C408 or C413 of murine SF-1. In yet a further embodiment, the invention provides a murine SF-1 receptor variant or active fragment thereof that includes the amino acid sequence SEQ ID NO: 19.

Further provided herein is a properly folded SF-1 receptor variant or active fragment thereof that has an amino acid sequence which encodes a SF-1 receptor or an active fragment thereof and lacks at least one of the following naturally occurring cysteine residues: a cysteine residue corresponding to C267 of murine SF-1; a cysteine residue corresponding to C302 of murine SF-1; and a cysteine residue corresponding to C423 of murine SF-1. Such a SF-1 receptor variant or active fragment thereof can exhibit increased monomer stability as compared to an analogous receptor or fragment which retains all cysteine residues lacking in the variant In one embodiment, the invention provides a SF-1 receptor variant or active fragment in which at least one naturally occurring cysteine residue is substituted with a non-cysteine residue. In another embodiment, the invention provides a SF-1 receptor variant or active fragment thereof having an amino acid sequence that lacks naturally occurring cysteine residues corresponding to C267 and C302 of murine SF-1. Such an amino acid sequence can have, for example, amino acid substitutions at cysteine residues corresponding to C267 and C302 of murine SF-1. In a further embodiment, the invention provides a SF-1 receptor variant or active fragment thereof having an amino acid sequence that lacks naturally occurring cysteine residues corresponding to C267 and C423 of murine SF-1. Such an amino acid sequence can have, for example, amino acid substitutions at cysteine residues corresponding to C267 and C423 of murine SF-1. The invention also provides a SF-1 receptor variant or active fragment thereof that lacks naturally occurring cysteine residues corresponding to C302 and C423 of murine SF-1. Such a SF-1 receptor variant or active fragment can have, for example, amino acid substitutions at cysteine residues corresponding to C302 and C423 of murine SF-1. In another embodiment, the invention provides a SF-1 receptor variant or active fragment thereof that lacks naturally occurring cysteine residues corresponding to corresponding to C267, C302 and C423 of murine SF-1. In such a variant or active fragment, useful amino acid sequences include, without limitation, those having amino acid substitutions at cysteine residues corresponding to C267, C302 and C423 of murine SF-1. In any of the above SF-1 receptor variants or active fragments which include amino acid substitutions, each of the substituted cysteine residues can be independently substituted with, for example, serine, threonine, alanine or valine. In other embodiments, the invention provides any of the above SF-1 receptor variants or active fragments in which each of the substituted cysteine residues is independently substituted with serine or threonine. In further embodiments, the invention provides any of the above SF-1 receptor variants or active fragments in which each of the substituted cysteine residues is substituted with serine.

An active fragment of the invention can include, for example, an SF-1 ligand-binding domain. In one embodiment, the invention provides a SF-1 active fragment containing SF-1 sequence limited to the ligand-binding domain. A variety of SF-1 receptors are useful in the invention including, without limitation, human and murine SF-1 receptors. In one embodiment, the invention provides a murine SF-1 receptor variant or active fragment thereof that includes the amino acid sequence SEQ ID NO: 15.

### BRIEF DESCRIPTION OF THE DRAWINGS

FIG. 1 shows a schematic of amino acid sequences and functional domains of steroidogenic factor-1 (SF-1) conserved among various species. The human, bovine, rat and mouse SF-1 sequences are illustrated, with the classic Cys₂-Cys₂ zinc finger DNA-binding domain (DBD), A box, hinge region, and ligand binding domain (LBD) indicated. The two major activation domains of SF-1, AF1 and AF2, also are shown as well as homologous regions R-II and R-III. Percentage homologies are indicated along with key amino acid numbers.

FIG. 2 shows a sequence alignment of the SF-1 hinge-ligand binding domain region with another member of the subclass V family of nuclear receptors, liver related homolog 1 (LRH-1). Included in the alignment are the amino acid sequences of the hinge-ligand binding domain of murine (SEQ ID NO: 1) and human (SEQ ID NO: 2) SF-1; murine (SEQ ID NO: 3). and human (SEQ ID NO: 4) LRH-1; and human retinoic X receptor α (RXRα; SEQ ID NO: 5). The amino acid numbering is indicated at the beginning of each line. Predicted α helices (twelve) and β-turns are indicated in bold and designated above the sequences.

FIG. 3 shows the nucleotide sequence (SEQ ID NO: 6) and corresponding amino acid sequence (SEQ ID NO: 7) of murine SF-1 available from Genbank accession NM_{—}139051 (version NM_{—}139051.1; see, also, Ikeda et al., Mol. Endocrin. 7:825 (1993).

FIG. 4 shows the nucleotide sequence (SEQ ID NO: 8) and corresponding amino acid sequence (SEQ ID NO: 9) of human SF-1 available from Genbank accession U76388 (version U76388.1).

FIG. 5 shows the nucleotide sequence (SEQ ID NO: 10) and corresponding amino acid sequence (SEQ ID NO: 11) of murine LRH-1 available from Genbank accession M81385 (version M81385.1).

FIG. 6 shows the nucleotide sequence (SEQ ID NO: 12) and corresponding amino acid sequence (SEQ ID NO: 13) of human LRH-1 available from Genbank accession NM_{—}003822 (version NM_{—}003822.2; see, also, Li et al., J. Biol. Chem. 273:29022-29031 (1998)).

FIG. 7 shows the nucleotide and corresponding amino acid sequences (residues 219-462) of four SF-1 variants each containing multiple cysteine to serine mutations. (A) The nucleotide and corresponding amino acid sequence of SF-1 Variant 1, which contains serine substitutions at cysteines C267, C302 and C423, are shown as SEQ ID NOS: 14 and 15, respectively. (B) The nucleotide and corresponding amino acid sequence of SF-1 Variant 2, which contains serine substitutions at cysteines C302 and C423, are shown as SEQ ID NOS: 16 and 17, respectively. (C) The nucleotide and corresponding amino acid sequence of SF-1 Variant 3, which contains serine substitutions at cysteines C408 and C413, are shown as SEQ ID NOS: 18 and 19, respectively. (D) The nucleotide and corresponding amino acid sequence of SF-1 Variant 4, which contains serine substitutions at cysteines C302, C408, C413 and C423, are shown as SEQ ID NOS: 20 and 21, respectively.

FIG. 8 shows a purification scheme for SF-1 ligand-binding domain and characterization of fractions after various purification steps. (A) A flow chart of steps for purification of SF-1 ligand-binding domain. This protocol includes three chromatography steps: TALON™, ion exchange, and gel filtration chromatography. (B) Schematic of the histidine-tagged SF-1(177-462) construct and removal of the 6XHIS-tag from the SF-1(177-462) protein by site-specific cleavage using the tobacco etch viral protease (TEV). A schematic of the bound and flow through fractions obtained using TALON™ chromatography also is shown. (C) SDS-PAGE electrophoresis of the SF-1(177-462) protein treated with increasing amounts of TEV protease. The 20 kDa TEV protease can be seen as a faint protein band running slightly below cleaved SF-1(177-462) protein (arrow). (D) Results obtained after the first TALON™ chromatography step (left panel), TEV cleavage and second TALON™ chromatography step (middle panel), and centrifugation to remove aggregates and minor contaminants (right panel). “B” indicates bound fraction; “FT” indicates the flow through fraction; “P” indicates pellet.

FIG. 9 shows the results of ion-exchange chromatography and gel filtration of SF-1(177-462). Left panel: A260 nM profile after cation-exchange chromatography (POROS-Q®). Right panel: A260 nM profile following gel filtration showing both a minor dimeric and major monomeric SF-1(177-462) species. Native polyacrylamide gel electrophoresis of the first peak (#1), and the second, lower molecular weight peak (#2) also is shown.

FIG. 10 shows reduction of SF-1(177-462) dimers by treatment with iodoacetate. Top panel: Mass spectroscopy on a Voyager DE (MALDI-TOF) instrument of aged SF-1(177-462) protein preparations. Lower panel: Mass spectroscopy of SF-1(177-462) preparations iodoacetylated prior to storage for a similar time period as the aged preparations shown in the top panel.

FIG. 11 shows a thermal denaturation curve for SF-1 Variant 3 generated using circular dichroism.

FIG. 12 shows dynamic light scattering analysis (DLS) of wild type SF-1 and several SF-1 variants. (A) DLS analysis of wild type SF-1(219-462). (B) DLS analysis of Variant 1. (C) DLS analysis of Variant 3.

FIG. 13 shows NMR analysis of SF-1 Variants 1 and 3.

FIG. 14 shows specific coactivator binding by several SF-1 receptor variants. (A) SF-1 Variant 1 binding to GRIP and SRC-like coactivator peptides. (B) Left panel: SF-1 Variant 3 binding to a GRIP coactivator peptide designated “LXXLL” and control peptide “LXXAA.” Right panel: SF-1 Variant 3 binding to SRC-like coactivator peptides designated “LXXLL” and control peptide “LXXAA.”

### DETAILED DESCRIPTION OF THE INVENTION

Steroidogenic factor-1 (SF-1), also known as adrenal 4-binding protein (Ad4BP) and officially designated NR5A1, is an essential factor in adrenal and gonadal development and for the proper functioning of the hypothalamic-pituitary-gonadal axis. SF-1 is a transcription factor which activates the promoters of various adrenal/gonadal steroid hydroxylase genes, as well as a variety of genes essential for endocrine organogenesis (Ikeda et al., Mol. Endocrinol. 7:852-860 (1993); Morohashi et al., Mol. Endocrinol. 7:1196-1204 (1993); and Parker and Schimmer, Endocr. Rev. 18:361-377 (1997)). Mammalian SF-1 exhibits significant similarity to Drosophila fushi tarazu factor 1 (Ftz-F1), a regulator of the developmental homeobox gene fushi tarazu (Lavorgna et al., Science 252:848-851 (1991); and Ueda et al., Genes Dev. 4:624-635 (1990)). The mouse SF-1 gene therefore has been designated Ftz-F1. SF-1 is conserved across both vertebrate and invertebrate species, indicating a conserved role for the protein in all metazoans (Honda et al., J. Biol. Chem. 268:7494-7502 (1993); Lala et al., Mol. Endocrinol. 6:1249-1258 (1992); Nomura et al., J. Biol. Chem. 270:7453-7461 (1995); Oba et al., Biochem. Biophys. Res. Comm. 226:261-267 (1996); Sun et al., Dev. Biol. 162:426-437 (1994); and Wong et al., J. Mol. Endocrinol. 17:139-147 (1996)). SF-1 homologs have been cloned, for example, from silkworm, chicken and frog as well as a variety of mammalian species.

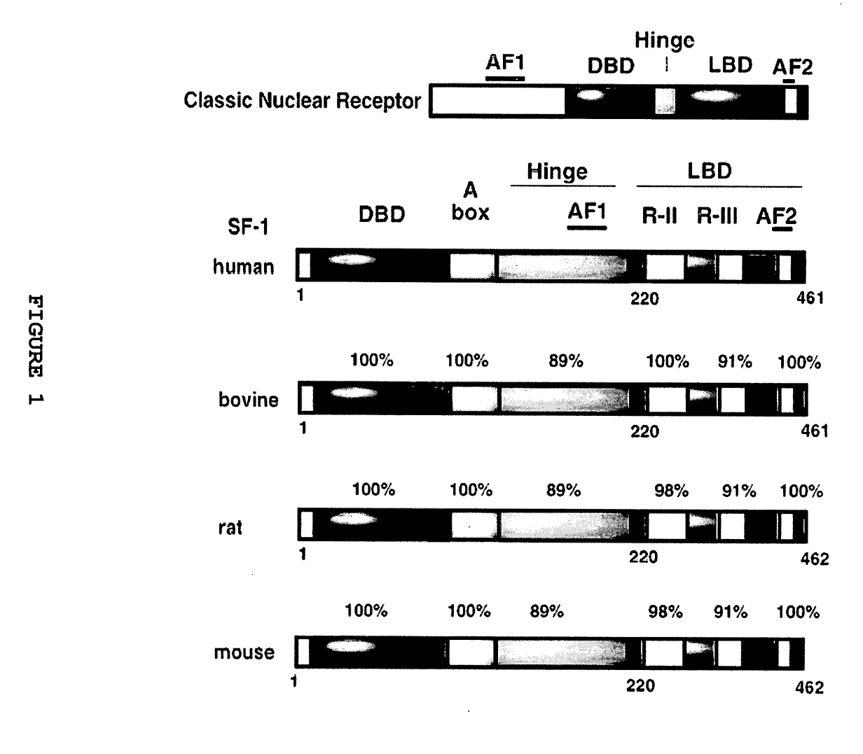

SF-1 is a member of the steroid receptor superfamily, and all SF-1 homologs have a common structural organization that shares several features with other members of the steroid receptor superfamily. A classic Cys₂-Cys₂ zinc finger DNA-binding domain (DBD) is present in the amino-terminal region; this domain confers high affinity binding to the SF-1 cognate response element (see FIG. 1) and is essential for DNA binding and subsequent transcriptional activation (and Wilson et al., Science 256:107-110 (1992); and Wilson et al., Mol. Cell. Biol. 13:5794-5804 (1993)). The major nuclear import signal also maps to the tandem zinc finger domain.

In contrast to the majority of steroid receptors, which function as dimers in DNA-binding and transcriptional regulation, SF-1 binds DNA as a monomer at an extended AGGTCA site such as the perfect SF-1 binding site, TCAAGGTCA (Wilson et al., supra, 1993). In SF-1 and other monomeric nuclear receptors, amino acid residues carboxy-terminal to the DNA-binding domain, denoted the “A” box, contribute to binding specificity by recognizing nucleotides 5′ to the AGGTCA response element, resulting in an extended monomer response element with increased binding fidelity (Ueda et al., Mol. Cell. Biol. 12:5667-5672 (1992); Wilson et al., supra, 1992; and Wilson et al., supra, 1993). Such monomeric nuclear receptors include liver related homolog 1/fetoprotein transcription factor (LRH-1/FTF/SF-1β), nerve growth factor-induced gene-B (NGF-IB), estrogen-related receptor 1 (ERR1), estrogen-related receptor 2 (ERR2) and retinoic acid receptor-related orphan nuclear receptor (ROR).

A variety of genes bound and regulated by SF-1 are known in the art. These SF-1 target genes include, for example, steroidogenic enzymes such as cytochrome P450 cholesterol side-chain cleavage enzyme (P450scc) and other steroidogenic targets such as the ACTH receptor; gonadal SF-1 target genes such as the gene for the male-specific Müllerian inhibiting substance (MIS), which is expressed in the Sertoli cells of the testis and responsible for regression of the female specific Müllerian duct; and pituitary and hypothalamic target genes such as αGSU and the luteinizing hormone β subunit (LHβ). A variety of additional SF-1 target genes are known in the art. See, for example, Hammer and Ingraham, Frontiers in Neurobiology 20:199-223 (1999).

Like other members of the steroid receptor superfamily, SF-1 contains a conserved ligand-binding domain positioned at the carboxy-terminus of the receptor and a conserved activation function 2 (AF2) sequence in the carboxy-terminal region of the ligand-binding domain. (see FIG. 1). In many nuclear receptors, this domain confers responsiveness to specific ligands that activate or, in some cases, repress receptor transcriptional activity (Evans, Science 240:889-895 (1988); Forman et al., Nature 395:612-615 (1998)). While SF-1-dependent transcriptional activity has been shown in one instance to exhibit a modest increase in response to 25-, 26-, and 27-hydroxycholesterol in CV-1 cells (Lala et al., Proc. Natl. Acad. Sci. USA 94:4895-4900 (1997)), a ligand for SF-1 has not been definitively identified, and SF-1 consequently is referred to as an “orphan receptor.”

SF-1 has been shown-to have transactivating activity in the absence of “exogenous” ligand. Two regions have been identified as important for SF-1 transactivation. Point mutations within the conserved AF2 hexamer motif, LLIEML (SEQ ID NO: 23), which is critical for transactivation function of many nuclear receptors (Mangelsdorf et al.., Cell 83:835-839 (1995)), abrogated SF-1 activity, as did removal of the distal hinge region that follows the DNA-binding domain. In contrast, much of the ligand-binding domain can be truncated without significantly impairing SF-1 transcriptional activity. Furthermore, in cell lines that support SF-1-transcriptional activity, the AF1 domain of SF-1 is constitutively phosphorylated on serine 203. A nonphosphorylatable mutant, SF-1^{S203A}, consistently exhibited a significant 50-80% reduction in transcriptional activity on the MIS promoter and other promoters as compared to wild-type SF-1 activity. Point mutations in the AF2 hexamer motif also resulted in significant reduction in SF-1 transactivation, and a further reduction in activity was observed when the AF2 hexamer mutation was combined with the S203A mutation (Hammer et al., Mol. Cell 3:521-526 (1999)). In sum, maximal SF-1 transcriptional activity requires both the AF1 in the distal hinge domain and AF2 (Crawford et al., Mol. Endocrinol. 11:1626-1635 (1997); Ito et al., Mol. Cell. Biol. 17:1476-1483 (1997)). Two motifs in particular, the phosphoSer²⁰³ and LLIEML (SEQ ID NO: 23) hexamer of the AF2 domain, are essential for full SF-1 transcriptional activity.

Consistent with a role for SF-1 as a regulator of steroid hydroxylases, SF-1 is expressed in the primary organs that produce steroid hormones, including adrenal cortical cells, testicular Leydig cells, and ovarian theca and granulosa cells (Ikeda et al., Mol. Endocrinol. 8:654-662 (1994); Sasano et al., J. Clin. Endocrinol. Metab. 80:2378-2380 (1995); Takayama et al., J.. Clin. Endocrinol. Metab. 80:2815-2821 (1995)). SF-1 also is expressed in the testicular Sertoli cell, the pituitary gonadotrope, and the ventral medial nucleus (VMN) of the hypothalamus (Asa et al., J. Clin. Endocrinol. Metab. 81:2165-2170 (1996); Hatano et al., Develop. 120:2787-2797 (1994); Ikeda et al., supra, 1994; Ingraham et al., Genes Dev. 8:2302-2312 (1994); Morohashi et al., Mol. Endocrinol. 7:1196-1204 (1993); and Roselli et al., Brain Res. Mol. Brain Res. 44:66-72 (1997)). SF-1 transcripts have been detected in spleen and placenta in addition to the gonad, adrenal, pituitary and hypothalamus.

In vivo significance of SF-1 has been demonstrated in SF-1 knockout mice. Homozygous Ftz-F1 −/− mice all died of glucocorticoid and mineralocorticoid insufficiency (Luo et al., Mol. Endocrinol. 9:1233-1239 (1995)). The absence of SF-1 resulted in female external genitalia regardless of chromosomal sex, consistent with a role for SF-1 in gonadal formation and synthesis of androgens such as dihydrotestosterone, which is required for development of male external genitalia. Gonads and adrenal glands were completely absent from both sexes. Furthermore, all mice, regardless of chromosomal sex, displayed a female internal reproductive tract (Luo et al., Cell 77:481-490 (1994); and Sadovsky et al., Proc. Natl. Acad. Sci. USA 92:10939-10943 (1995)), consistent with a known role of SF-1 in regulation of Müllerian inhibiting substance (Giuili et al., Development 124:1799-1807 (1997); Shen et al., Cell 77:651-661 (1994)). In the absence of this inhibitory substance, regression of the Müllerian duct, the precursor of the vagina, uterus and fallopian tube, does not take place. SF-1 null mice also lacked follicle stimulating hormone (FSH) and luteinizing hormone (LH) expression in the anterior pituitary. These results indicate that SF-1 is critical for appropriate development of the adrenals, gonads and pituitary gonadotropes.

The phenotype of the SF-1 null mice parallels the phenotype observed in the human syndrome of X-linked congenital hypoplasia, a disorder which is characterized by hypoplastic adrenal glands often accompanied by profound hypogonadism. The gene responsible for the human syndrome, DAX-1 (dosage-sensitive sex reversal-adrenal hypoplasia congenita critical region on the X chromosome), localizes to Xp21 and, like deletions of SF-1, DAX-1 deletions result in profound adrenal hypoplasia in humans (Muscatelli et al., Nature 372:672-676 (1994); and Zanaria et al., Nature 372:635-641 (1994)). Dax-1 also is an orphan nuclear steroid receptor expressed in multiple endocrine organs; Dax-1 and SF-1 appear to colocalize to cells of the adrenals, gonads, gonadotropes and VMN (Ikeda et al., Mol. Endocrinol. 9:478-486 (1995); Swain et al., Nat. Genetics 12:404-409 (1996)). Together with the similar phenotypes of SF-1 null mice and Dax mutations in humans, these results reinforce the importance of SF-1 and indicate that SF-1 and Dax-1 can work together as essential regulators of the hypothalamic-pituitary-steroidogenesis axis in humans.

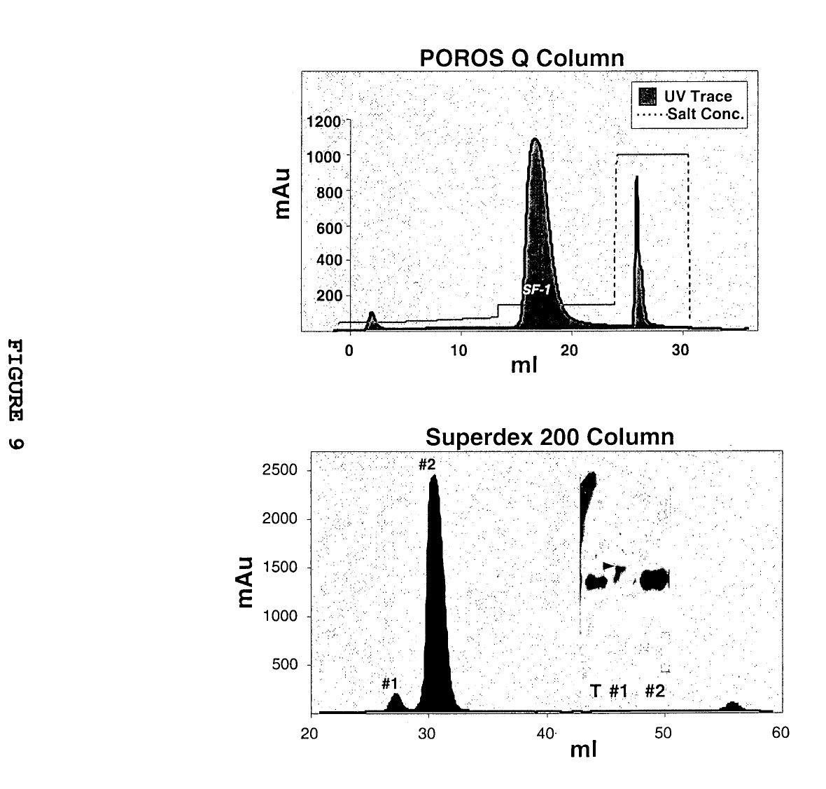

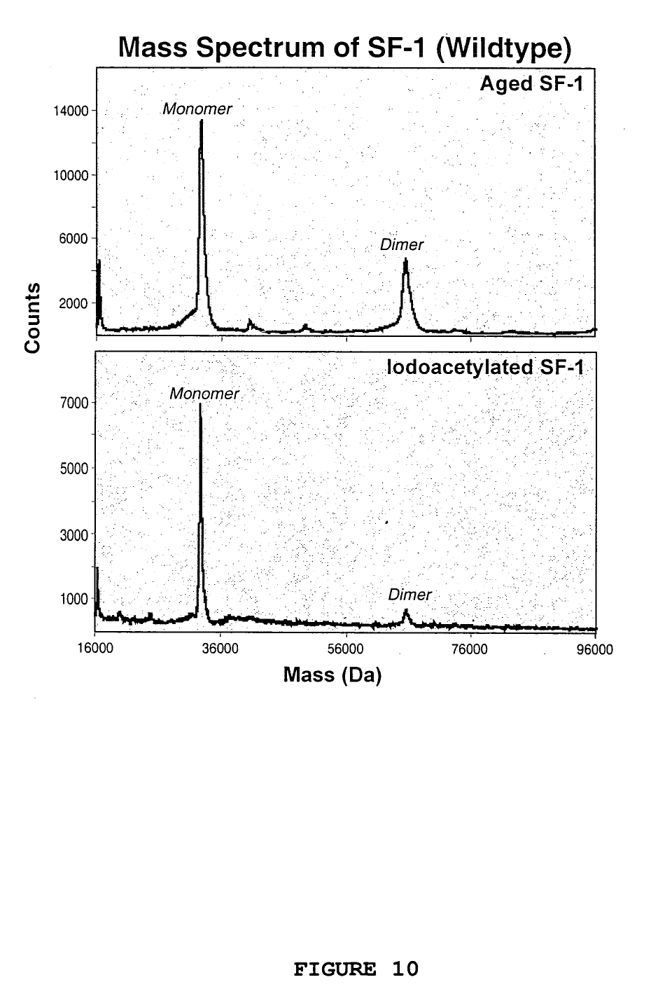

As disclosed herein, freshly produced protease-cleaved SF-1(177-462) was predominantly monomeric (FIG. 9) but exhibited an equilibrium which favored the dimeric form over time. As shown herein in FIG. 10, upper panel, mass spectroscopy demonstrated significant dimer formation in aged SF-1(177-462) preparations. However, as further disclosed herein, SF-1(177-462) protein preparations were iodoacetylated to covalently modify surface cysteines; monomer stability was significantly enhanced in iodoacetylated SF-1 preparations as shown herein in FIG. 10, lower panel.

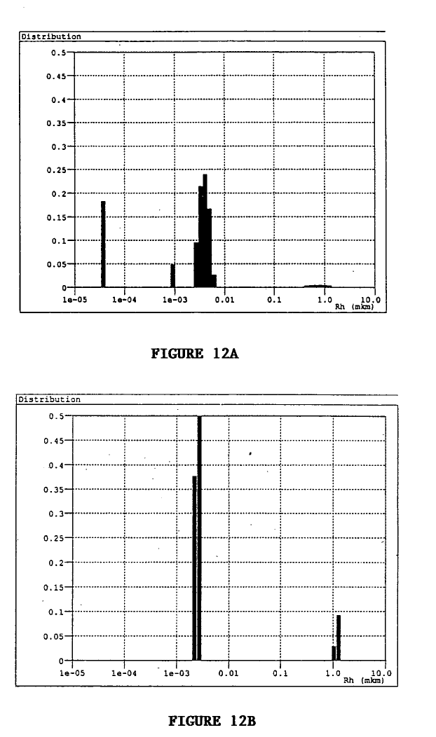

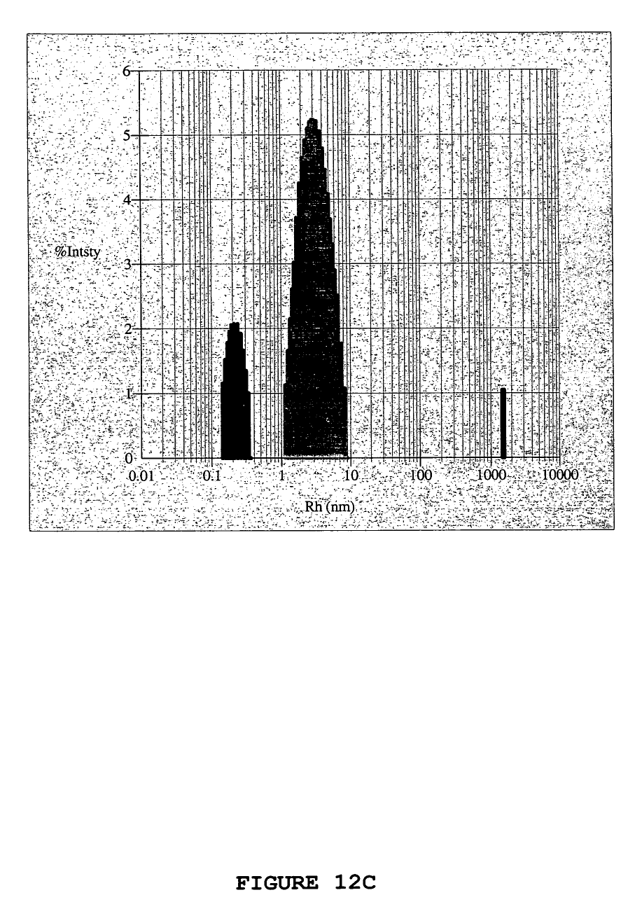

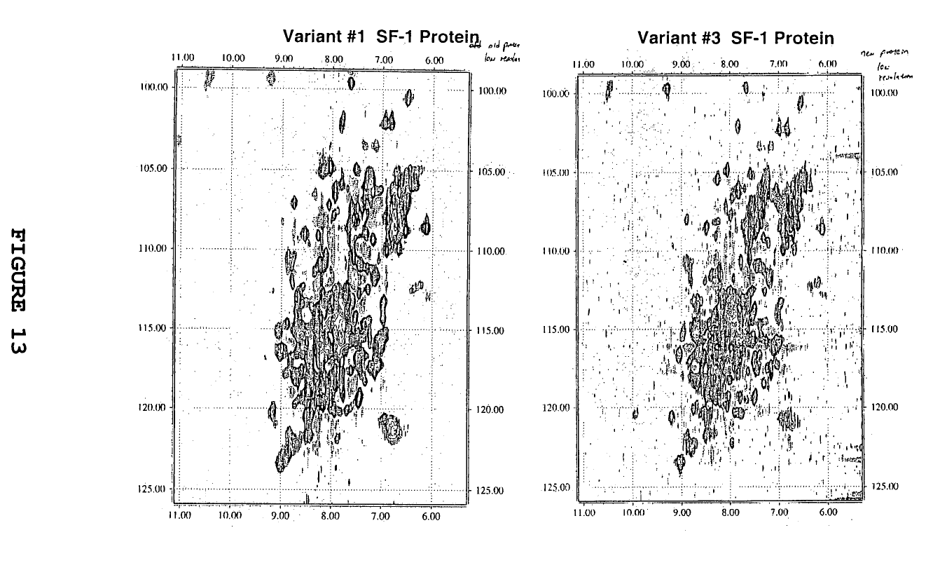

As additionally disclosed herein in Example III, mass spectroscopy of tryptic fragments of iodoacetylated murine SF-1, and three dimensional modeling of the SF-1 ligand binding domain were used to identify cysteines C267, C302, C408, C413 and C423 as potentially solvated residues available for intermolecular disulfide bond formation. Furthermore, two different SF-1 mutants containing multiple serine for cysteine substitutions were prepared; Variant 1, which contains residues 219-462 with serine substitutions at C267, C302 and C423, and Variant 3, which contains residues 219-462 with serine substitutions at C408 and C413 (see Example IV). As shown herein in FIGS. 12 and 13, these two murine SF-1 variants exhibited a significantly reduced tendency to dimerize relative to wild type murine SF-1.

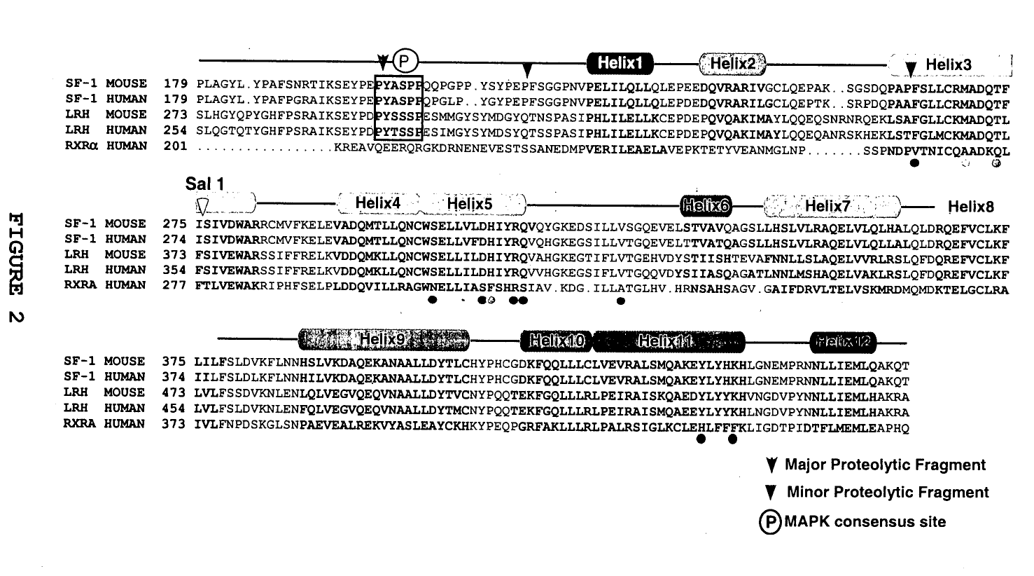

These results indicate that a variant of SF-1 can exhibit increased monomer stability and reduced aggregation as compared to the analogous receptor that retains the cysteine residues lacking in the variant. Based on the high degree of conservation between the ligand-binding domains of SF-1 and LRH-1 illustrated, for example, in FIG. 2, these results further indicate that variants of LRH-1 which are similarly modified to remove one or more potentially solvated cysteine residues also can be properly folded and can further exhibit increased monomer stability as compared to the analogous wild type LRH-1 receptor. Such SF-1 or LRH-1 variants can be useful, for example, in any functional or structural studies that rely on relatively high concentrations of protein. As an example, such variants can be useful in protein crystallization studies, which generally rely on protein preparations that are at least 80% monomeric.

Thus, the present invention provides a properly folded SF-1-like receptor variant, or active fragment thereof, which has an amino acid sequence that encodes a SF-1-like receptor or an active fragment thereof and that lacks at least one naturally occurring cysteine residue within the ligand-binding-domain of the receptor. Such a properly folded SF-1 receptor variant or active fragment thereof can exhibit increased monomer stability as compared to the analogous receptor that retains the cysteine residues lacking in the variant. In one embodiment, at least one naturally occurring cysteine residue within the ligand-binding domain of the SF-1-like receptor is substituted with a non-cysteine residue.

A SF-1-like receptor variant or active fragment of the invention can be, for example, a SF-1 receptor variant or a LRH-1 receptor variant, or an active fragment thereof. An active fragment of the invention can include, for example, the ligand-binding domain. In one embodiment, the invention provides an active fragment containing a SF-1-like receptor variant ligand-binding domain without additional sequence from the same receptor. In another embodiment, the invention provides an active fragment containing a ligand-binding domain and activation function 1 (AF1 ) of a SF-1-like receptor. In a further embodiment, the active fragment contains a ligand-binding domain and AF1 without additional sequence from the same receptor.

A SF-1-like receptor variant or active fragment of the invention lacks at least one naturally occurring cysteine residue within the ligand-binding domain of the receptor. In one embodiment, the invention provides a SF-1-like receptor variant, or active fragment thereof, which lacks at least two naturally occurring cysteine residues within the ligand-binding domain of the receptor. In another embodiment, the invention provides a SF-1-like receptor variant or active fragment thereof, in which at least two naturally occurring cysteine residues within the ligand-binding domain are substituted with non-cysteine residues. In a further embodiment, the invention provides a SF-1-like receptor variant, or active fragment thereof, which lacks at least three naturally occurring cysteine residues within the ligand-binding domain of the receptor. If desired, at least three naturally occurring cysteine residues within the ligand-binding domain of the SF-1-like receptor variant can be substituted with non-cysteine residues.

The term “variant,” as used herein, means a SF-1-like receptor or other monomeric nuclear receptor, as described further below, having a non-naturally occurring amino acid sequence. A receptor variant of the invention has a ligand-binding domain in which one or more cysteine residues is deleted or substituted as compared to the amino acid sequence of the analogous wild type receptor.

A SF-1-like receptor variant of the invention is properly folded. As used herein, the term “properly folded” means a defined and homogeneous two- and three-dimensional structure. A properly folded receptor variant generally has the same two- and three-dimensional structure that characterizes the analogous wild type receptor. Proper folding can be determined by any of a variety of routine techniques, including a well-defined melting point transition. Thermal denaturation curves can be generated, if desired, using circular dichroism as disclosed herein.

In one embodiment, a SF-1-like receptor variant exhibits increased monomer stability as compared to the analogous receptor that retains the cysteine residues lacking in the variant. Monomers are proteins formed from a single polypeptide chain; dimers are formed from the association of two monomers. As used herein, the term “increased monomer stability” means that, in a purified protein preparation at high concentration, a higher percentage of monomeric protein is present as compared to the percentage of monomeric protein present in a purified protein preparation of analogous receptor that retains the cysteine residues lacking in the variant and is assayed under the same conditions. The term “analogous receptor,” as used herein, means the naturally occurring receptor having a sequence exhibiting the most similarity to the receptor variant. An analogous receptor may or may not have a wild type sequence and, furthermore, refers, as appropriate, to an active fragment of receptor. Thus, wherein one or more cysteine substitutions is made in a murine SF-1 fragment having residues 219 to 462, the analogous receptor is a fragment of wild type murine SF-1 having residues 219 to 462. It is understood that, other than lacking one or more naturally occurring cysteines, the receptor variant has an identical sequence and is assayed under the same conditions as the “analogous receptor.”

The dimerization observed with aged preparations of wild type SF-1(177-462), for example, in FIG. 10, occurs at the relatively high protein concentrations used in many in vitro studies. In general, dimerization or other aggregation of wild type SF-1 ligand-binding domain generally is observed when purified SF-1 ligand-binding domain concentrations above 30 μM are used. Thus, it is understood that “increased monomer stability” is assayed at relatively high protein concentrations of the receptor variant such as concentrations of at least 30 μM, for example, 50 to 100 μM.

Methods for assaying increased monomer stability include, without limitation, dynamic light scattering (DLS) analysis. In DLS analysis, the diffusion constant of molecules moving randomly in solution is calculated from the autocorrelation function of the scattered light. The very small signal values generally are collected using a solid state photon counter (avalanche photo diode) and analyzed using autocorrelator electronics incorporating high-speed digital signal processors. From the diffusion constant, the hydrodynamic radius is calculated using the Stokes-Einstein equation.

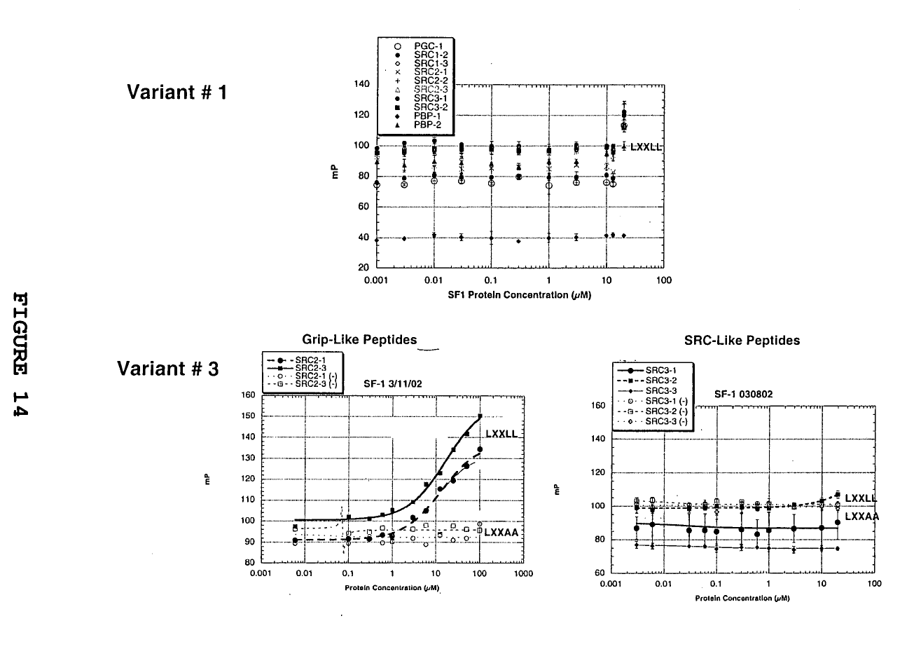

As further disclosed herein, a properly folded SF-1-like receptor variant or active fragment thereof can retain one or more functions of the analogous wild type receptor. Such functions include, without limitation, ligand-binding and co-activator binding. As a non-limiting example, Variant 3 functioned to specifically bind GRIP coactivator peptides as shown in FIG. 14. These results indicate that SF-1-like receptor variants can retain one or more wild type functions despite having a non-naturally occurring amino acid sequence.

As used herein, the term “SF-1-like receptor” means a SF-1 receptor or LRH-1 receptor. An SF-1-like receptor is a protein having a ligand binding domain with at least about 55% identity to the ligand binding domain of murine SF-1 (residues 219 to 462 of SEQ ID NO: 1) or with at least about 55% identity to the ligand binding domain of murine LRH-1 (residues 296 to 542 of SEQ ID NO: 3). An SF-1-like receptor can have, for example, at least 65%, 70%, 75%, 80%, 85%, 90%, 95%, 96%, 97%, 98% or 99% identity to the ligand binding domain of murine SF-1 (residues 219 to 462 of SEQ ID NO: 1) or at least 65%, 70%, 75%, 80%, 85%, 90%, 95%, 96%, 97%, 98% or 99% identity to the ligand binding domain of murine LRH-1 (residues 296 to 542 of SEQ ID NO: 3).

The invention relates, in part, to a SF-1 receptor variant or active fragment thereof. As used herein, the term “SF-1” is synonymous with “steroidogenic factor-1” and means an SF-1-like receptor that is more closely related to murine SF-1 (SEQ ID NO: 1) than to murine LRH-1 (SEQ ID NO: 3). An SF-1 receptor generally is a protein having a ligand binding domain with at least about 65% identity to the ligand binding domain of murine SF-1 (SEQ ID NO: 1). The term SF-1 encompasses a variety of species homologs, including human, primate and other mammalian homologs of SF-1 such as bovine, horse, pig, rat and murine SF-1 homologs as well as other vertebrate and invertebrate homologs. The amino acid and encoding nucleic acid sequences for such homologs are well known in the art. As non-limiting examples, a human SF-1 coding sequence is available from Genbank accession U76388; a bovine SF-1 coding sequence is available from Genbank accession D13569 (Honda et al., J. Biol. Chem. 268:7494-7502 (1993)); a horse SF-1 coding sequence is available from Genbank accession AF203911 (Boerboom et al., Endocrinology 141:4647-4656 (2000)); a pig SF-1 coding sequence is available from Genbank accession U84399 (Pilon et al., Endocrinology 139:3803-3812 (1998)); and a murine SF-1 coding sequence is available from Genbank accession NM_{—}139051.1. The coding sequences of additional SF-1 homologs also are well known in the art; examples include, but are not limited to, the coding sequence of chicken SF-1 available from Genbank accession AB002404 (Kudo and Sutou, Gene 197:261-268 (1997)) and the coding sequence of frog SF-1 available from Genbank accession AB017352.1 (Kawano et al., Gene 222:169-176 (1998)). The skilled person understands that the coding sequences of a variety of other SF-1 homologs are known in the art or can be identified by routine methods and that a variant or active fragment of the invention can be prepared from any such SF-1 homolog.

The invention also relates, in part, to a LRH-1 receptor variant or active fragment thereof. As used herein, the term “LRH-1” is synonymous with “liver related homolog 1,” “fetoprotein transcription factor” and “SF-1β” and means an SF-1-like receptor that is more closely related to murine LRH-1 (SEQ ID NO: 3) than to murine SF-1 (SEQ ID NO: 1). A LRH-1 receptor generally is a protein having a ligand binding domain with at least about 65% identity to the ligand binding domain of murine LRH-1 (SEQ ID NO: 3), shown herein in FIG. 2. The term LRH-1 encompasses a variety of species homologs, including human, primate and other mammalian homologs of LRH-1 such as bovine, horse, pig, rat and murine LRH-1 homologs as well as other vertebrate and invertebrate homologs. The amino acid and encoding nucleic acid sequences for such homologs are well known in the art. As non-limiting examples, a human LRH-1 coding sequence is available from Genbank accession NM_{—}003822.2 (Li et al., J. Biol. Chem. 273:29022-29031 (1998)); a rat LRH-1 coding sequence is available from Genbank accession NM_{—}021742; a murine LRH-1 coding sequence is available from Genbank accession M81385; and a chicken LRH-1 coding sequence is available from Genbank accession AB002403. The skilled person understands that the coding sequences of a variety of other LRH-1 homologs are known in the art and that routine molecular methods can be used to identify additional homologs. A LRH-1 receptor variant or active fragment of the invention can be prepared from any such homolog.

A SF-1-like receptor variant or other monomeric nuclear receptor variant or active fragment of the invention has an amino acid sequence which lacks at least one naturally occurring cysteine residue within the ligand-binding domain. Such a receptor variant or active fragment of the invention also can have one or more additional amino acid sequence alterations as compared to the wild type receptor sequence. Thus, in addition to lacking one or more naturally occurring cysteine residues within the ligand-binding domain, a variant or active fragment of the invention can have one or more additions, deletions or substitutions that confer, for example, altered in vitro or in vivo stability, solubility, bioavailability, ligand binding activity or coactivator binding activity or other favorable characteristic or that facilitate, for example, expression, purification or detection of the variant or active fragment thereof. Such a modification can be deliberate, as through site-directed mutagenesis, or can be accidental, as through mutation in hosts harboring an encoding nucleic acid, and can be located within the receptor ligand-binding domain or outside of this domain. All such modified receptors and active fragments are included in the definition of a receptor variant as long as the variant or active fragment has an amino acid sequence which lacks at least one naturally occurring cysteine residue within the ligand-binding domain and as long as the variant or active fragment retains at least one biological activity of the analogous wild type receptor. Such a biological activity can be, for example, ligand binding; coactivator binding such as GRIP binding; or transactivation.

Thus, the term “SF-1-like receptor variant” encompasses polypeptides having an amino acid sequence which, other than lacking one or more cysteine residues within the ligand-binding domain, is identical to the analogous wild type receptor sequence, as well as polypeptides having a similar, non-identical sequence. Minor modifications of murine or human SF-1 (SEQ ID NO: 7 or 9) that do not destroy transactivation activity or the ability to associate with a coactivator such as GRIP fall within the definition of a SF-1 receptor variant when combined with the deletion or substitution of at least one naturally occurring cysteine residue within the ligand-binding domain. Similarly, minor modifications of LRH-1 that do not destroy transactivation activity or the ability to associate with a coactivator such as GRIP fall within the definition of an LRH-1 receptor variant when combined with the deletion or substitution of at least one naturally occurring cysteine residue within the ligand-binding domain.

It further is understood that various moieties can be attached to a SF-1 receptor variant, LRH-1 receptor variant or another SF-1-like receptor variant, monomeric receptor variant or active fragment of the invention. As an example, a variety of heterologous tags are well known in the art and readily available and can be included in a receptor variant or active fragment of the invention. Such tags include epitope tags and encompass, without limitation, the V5 tag, the FLAG tag DYKDDDDK (SEQ ID NO: 24); the HA tag YPYDVPDYA (SEQ ID NO: 25); the c-Myc epitope EQKLISEEDL (SEQ ID NO: 26); the AU1 tag DTYRYI (SEQ ID NO: 27); and the 6-HIS tag. One skilled in the art understands that these and other heterologous tags can be fused to a receptor variant or active fragment thereof to conveniently purify or detect the fused protein. A receptor variant, or active fragment of the invention also can be fused, for example, to a heterologous DNA-binding domain, or heterologous activation domain, in place of the native DNA-binding or activation domain to form a chimeric receptor variant. Additional moieties that can be attached to a receptor variant include heterologous polypeptides or polypeptide domains, peptides, carbohydrates, lipids, radionuclides and fluorescent and other chemical moieties. These fusion polypeptides or polypeptide conjugates also are encompassed by the invention.

Also provided herein is a monomeric nuclear receptor, or active fragment thereof, containing an amino acid sequence that encodes a monomeric nuclear receptor or an active fragment thereof and lacks at least one naturally occurring cysteine residue within the ligand-binding domain of the receptor.

A nuclear receptor can be identified through primary, secondary or tertiary structural homology to at least one ligand-binding domain of a known nuclear hormone receptor such as a retinoid receptor, glucocorticoid receptor, estrogen receptor, progesterone receptor, thyroid receptor, vitamin D receptor, mineralocorticoid receptor or another member of the steroid hormone receptor superfamily such as those described in Whitfield et al., J. Cell. Biochem. 75 Suppl. 32:110-122 (1999). A nuclear receptor generally has a tertiary structure which is a sandwich of 11 to 13 α-helices and several small β-strands organized around a lipophilic binding cavity (Williams and Sigler, Nature 393:392-396 (1998)). The ligand-binding domain of a nuclear receptor generally contains three subregions: a conserved E1 domain; the heptad 9 (h9) subregion; and an AF2 subregion. Such a nuclear receptor can be recognized, in part, by conserved lysine, phenylalanine and aspartic acid-glutamine residues in the E1 subregion (Whitfield, supra, 1999).

As used herein, the term “monomeric nuclear receptor” means a nuclear receptor that specifically binds DNA as a monomer with high affinity. Such a monomeric nuclear receptor generally binds to a cognate binding site with an affinity of at least about 100 nM (Shen et al., Cell 77: 651-661 (1994)). Examples of monomeric nuclear receptors include but are not limited to SF-1, LRH-1, NGF-IB, ERR1, ERR2, ROR, Rev-ErbA and Rev-ErbB and other nuclear receptors with similar properties (Lazar and Harding, “Monomeric Nuclear Receptors” in Freedman (Ed.), Molecular Biology of Steroid and Nuclear Hormone Receptors Boston: Birkhauser pp. 261-279 (1998)). In one embodiment, the invention provides a monomeric nuclear receptor, or active fragment thereof, other than ROR.

It is clear that a variety of cysteine substitutions can be useful in the SF-1-like receptor variants and active fragments of the invention. In particular embodiments, the ligand-binding domain of a SF-1-like receptor or active fragment thereof contains substitutions of at least one, two or three naturally occurring cysteine residues, where each of the cysteine residues is independently substituted with serine, threonine, alanine, valine, glycine, leucine, isoleucine, tyrosine, phenylalanine, tryptophan, methionine or histidine. In further embodiments, at least one, two or three naturally occurring cysteine residues each is independently substituted with serine or threonine. In still further embodiments, at least one, two or three naturally occurring cysteine residues is substituted with serine.

The invention also provides a SF-1-like receptor variant, such as a SF-1 or LRH-1 receptor variant, or an active fragment thereof, which contains amino acid substitutions at cysteine residues corresponding to C267, C302 and C423 of murine SF-1. In one embodiment, the invention provides a SF-1 receptor variant, or active fragment thereof, containing amino acid substitutions at cysteine residues corresponding to C267, C302 and C423 of murine SF-1. Such a SF-1 receptor variant can be, for example, a human SF-1 receptor variant. In another embodiment, the invention provides a SF-1 receptor variant, or active fragment thereof, containing serine substitutions at cysteine residues corresponding to C267, C302 and C423 of murine SF-1. In a further embodiment, the invention provides a human SF-1 receptor variant, or active fragment thereof, containing serine substitutions at cysteine residues corresponding to C267, C302 and C423 of murine SF-1.

The invention further provides a SF-1-like receptor variant, such as a SF-1 or LRH-1 receptor variant, or an active fragment thereof, which contains amino acid substitutions at cysteine residues corresponding to C302 and C423 of murine SF-1. In one embodiment, the invention provides a SF-1 receptor variant, or active fragment thereof, containing amino acid substitutions at cysteine residues corresponding to C302 and C423 of murine SF-1. In another embodiment, the invention provides a human SF-1 receptor variant, or active fragment thereof, containing amino acid substitutions at cysteine residues corresponding to C302 and C423 of murine SF-1. In another embodiment, the invention provides a SF-1 receptor variant, or active fragment thereof, that contains serine substitutions at cysteine residues corresponding to C302 and C423 of murine SF-1. In yet a further embodiment, the invention provides a human SF-1 receptor variant, or active fragment thereof, that contains serine substitutions at cysteine residues corresponding to C302 and C423 of murine SF-1.

The invention additionally provides a SF-1-like receptor variant, such as a SF-1 or LRH-1 receptor variant, or an active fragment thereof, which contains amino acid substitutions at cysteine residues corresponding to C408 and C413 of murine SF-1. In one embodiment, the invention provides a SF-1 receptor variant, or active fragment thereof, containing amino acid substitutions at cysteine residues corresponding to C408 and C413 of murine SF-1. In another embodiment, the invention provides a human SF-1 receptor variant, or active fragment thereof, containing amino acid substitutions at cysteine residues corresponding to C408 and C413 of murine SF-1. In a further embodiment, the invention provides a SF-1 receptor variant, or active fragment thereof, containing serine substitutions at cysteine residues corresponding to C408 and C413 of murine SF-1. As a non-limiting example, such a SF-1 receptor variant can be a human SF-1 receptor variant.

In the receptor variants and active fragments of the invention, one or more cysteines can be substituted with alternative amino acids. It is understood that a naturally occurring cysteine residue can be substituted with any amino acid; a naturally occurring cysteine residue can be substituted, if desired, with an amino acid of similar size and polarity. Cysteine is most similar in size and polarity to serine, and also is similar in size and polarity to threonine, alanine and valine.

It is understood that any number of naturally occurring cysteines within the ligand-binding domain of SF-1, LRH-1 or another SF-1-like receptor or monomeric nuclear receptor can be depleted by deletion or substitution to produce a receptor variant or active fragment of the invention. A SF-1-like receptor variant or active fragment of the invention can lack, for example, a single naturally occurring cysteine, or can lack two or more, three or more, four or more, five or more, six or more, or seven or more cysteines that occur naturally within the ligand-binding domain, for example, two, three, four, five, six, seven or eight cysteines that occur naturally within the ligand-binding domain. Furthermore, where two or more naturally occurring cysteines are depleted from the receptor or active fragment thereof, they can be depleted in any combination. It also is understood that, where multiple naturally occurring cysteines are depleted, both deletion and substitution can be used to remove different naturally occurring cysteine residues in the same receptor variant or active fragment thereof.

As can be seen in FIG. 2, eight cysteine residues occur naturally in the ligand-binding domain of a SF-1 receptor; these cysteines correspond to C248, C267, C284, C302, C371, C408, C413 and C423 in murine SF-1. Thus, a SF-1 variant or active fragment thereof can lack one or more cysteines corresponding to C248, C267, C284, C302, C371, C408, C413 or C423 or can lack, for example, any combination of two or more, three or more, four or more, five or more, six or more, or seven or more cysteines corresponding to C248, C267, C284, C302, C371, C408, C413 or C423. In one embodiment, a SF-1 variant or active fragment thereof lacks a single naturally occurring cysteine within the ligand-binding domain, where this single cysteine is C248, C267, C284, C302, C371, C408, C413 or C423.

As disclosed herein, solvated cysteines that can participate in intermolecular disulfide bond formation were identified by determining the cysteines most highly reactive with iodoacetamide and by modeling the three-dimensional model of the murine SF-1 ligand-binding domain using the crystal structure of liganded human RXRα (Egea et al., EMBO J. 19:2592-2601 (2000)). In particular, C267, C302 and C423 were identified by reactivity with iodoacetamide, and C408 and C413 were identified by three-dimensional modeling. Thus, a SF-1 receptor variant or active fragment of the invention can have, for example, a substitution at a single naturally occurring cysteine residue within the ligand-binding domain, where the single substituted cysteine residue corresponds to C267, C302, C408, C413 or C423 in murine SF-1. A SF-1 receptor variant or active fragment of the invention also can contain, for example, substitutions at two naturally occurring cysteine residues within the ligand-binding domain, where the two cysteine residues correspond to C267 and C302, C267 and C408, C267 and C413, C267 and C423, C302 and C408, C302 and C413, C302 and C423, C408 and C413, C408 and C423, or C413 and C423, in murine SF-1. As discussed further below, a variety of residues, including, but not limited to, serine, threonine, alanine, and valine, can be independently substituted for naturally occurring cysteine residues.

Of the potentially solvated cysteines in the SF-1 ligand-binding domain, four cysteines, those corresponding to C267, C302, C371 and C408 in murine SF-1, are present in the LRH-1 receptor. Thus, a LRH-1 receptor variant or active fragment of the invention can have, for example, a substitution at a single naturally occurring cysteine residue within the ligand-binding domain, where the single cysteine residue corresponds to C267, C302, C371 or C408 in murine SF-1. A LRH-1 receptor variant or active fragment of the invention also can have, for example, substitutions for two naturally occurring cysteine residues within the ligand-binding domain, where the two cysteine residues correspond to C267 and C302, C267 and C371, C267 and C408, C302 and C371, C302 and C408, or C371 and C408, in murine SF-1. It is understood that any of a variety of amino acid substitutions can be useful in a LRH-1 variant or active fragment of the invention, including, without limitation, serine, threonine, alanine, valine and the other neutral amino acids described hereinbelow.

In one embodiment, the invention provides a SF-1, LRH-1 or other SF-1-like receptor variant having a ligand-binding domain in which at least one, two or three naturally occurring cysteines is independently substituted with serine, threonine, alanine, valine, glycine, leucine, isoleucine, tyrosine, phenylalanine, tryptophan, methionine or histidine. In a further embodiment, each of the substituted cysteines in the receptor variant or active fragment thereof is independently substituted with serine, threonine, alanine or valine. In yet a further embodiment, each of the substituted cysteines in the receptor variant or active fragment thereof is independently substituted with serine or threonine. In another embodiment, each of the substituted cysteines in the receptor variant or active fragment thereof is substituted with serine.

As used herein in reference to the substitution of two or more naturally occurring cysteine within a ligand-binding domain, the term “independently” means that the amino acids selected to substitute for the naturally occurring cysteines can be the same or different. As an example, the invention encompasses a SF-1-like receptor variant, or active fragment thereof, which has a serine for cysteine substitution at the cysteine corresponding to C267, an alanine for cysteine substitution at the cysteine corresponding to C302, and a valine for cysteine substitution at the cysteine substitution corresponding to C423. Similarly, the invention encompasses a SF-1-like receptor variant, or active fragment thereof, which has a threonine for cysteine substitution at the cysteine corresponding to C302 and serine for cysteine substitution at the cysteine corresponding to C423, or which has serine for cysteine substitutions at both C408 and C413.

It is understood that, in different species homologs and isotypes and in different receptors, the residue numbering of a particular cysteine may vary; specific cysteine residues are defined herein with reference to the murine SF-1 amino acid sequence shown in FIG. 2. As used herein, the term “corresponding to” means the cysteine residue in a different receptor or species homolog that occupies, relative to the reference cysteine, the analogous position in the primary, secondary or tertiary structure of the receptor. Such a “corresponding” cysteine residue can be readily identified by routine methods, for example, alignment of primary sequences or assignment of conserved secondary structures. As an example, cysteine 267 in murine SF-1 corresponds to cysteine 266 in human SF-1, to cysteine 365 in murine LRH-1, and to cysteine 346 in human LRH-1. A cysteine corresponding to C267 also can be identified, for example, as a cysteine positioned within helix 3. Similarly, a cysteine corresponding to C302 can be identified by its position between helices 4 and 5; a cysteine corresponding to C408 can be identified by its position at the C-terminus of helix 9; a cysteine corresponding to C413 can be identified by its position between helices 9 and 10; and a cysteine corresponding to C423 can be identified by its position between helices 10 and 11.

The present invention further provides a nucleic acid molecule which encodes a SF-1-like receptor variant, or active fragment thereof, having an amino acid sequence lacking at least one naturally occurring cysteine residue within the ligand-binding domain. The invention further provides vectors and related host cells that contain a nucleic acid molecule encoding a SF-1-like receptor variant or active fragment of the invention. In one embodiment, the vector is a bacterial expression vector.

A nucleic acid molecule of the invention can encode any of the SF-1-like receptor variants or active fragments of the invention, including receptor variants and active fragments that exhibit increased monomer stability at high concentration. A nucleic acid molecule of the invention can encode, for example, a SF-1 receptor variant or active fragment thereof, or a LRH-1 receptor variant or active fragment thereof. The active fragment can include the ligand-binding domain, with or without additional receptor sequence, or can contain the ligand-binding domain and AF1, with or without additional sequence. It is understood, therefore, that an active fragment can be a larger fragment encompassing the ligand binding domain, or encompassing both the ligand-binding domain and AF1.

In particular embodiments, a nucleic acid molecule of the invention encodes a SF-1-like receptor variant or active fragment thereof which lacks at least one, two or three cysteine residues within the ligand-binding domain of the receptor. In additional embodiments, the invention provides a nucleic acid molecule encoding a SF-1-like receptor variant or active fragment thereof, in which at least one, two, or three cysteine residues within the ligand-binding domain are substituted with non-cysteine residues. In a further embodiment, the one or more substituted cysteines are independently substituted with serine, threonine, alanine, valine, glycine, leucine, isoleucine, tyrosine, phenylalanine, tryptophan, methionine or histidine. In another embodiment, the one or more substituted cysteines are independently substituted with serine or threonine.

Further provided by the invention is a nucleic acid molecule encoding a SF-1 or LRH-1 receptor variant, or an active fragment thereof, which contains amino acid substitutions at cysteine residues corresponding to C267, C302 and C423 of murine SF-1. Such a nucleic acid molecule can have, for example, serine substitutions at the cysteine residues corresponding to C267, C302 and C423 of murine SF-1. The invention also provides a nucleic acid molecule encoding a SF-1 or LRH-1 receptor variant, or an active fragment thereof, which contains amino acid substitutions at cysteine residues corresponding to C302 and C423 of murine SF-1. In such a nucleic acid molecule, the cysteine residues corresponding to C302 and C423 of murine SF-1 can be substituted, for example, with serine. The invention further provides a nucleic acid molecule encoding a SF-1 or LRH-1 receptor variant, or an active fragment thereof, which contains amino acid substitutions at cysteine residues corresponding to C408 and C413 of murine SF-1. In one embodiment, the nucleic acid molecule encodes a SF-1 or LRH-1 receptor variant or active fragment thereof containing serine substitutions at cysteine residues corresponding to C408 and C413 of murine SF-1.

As used herein, the term “nucleic acid molecule” means any polymer of two or more nucleotides, which are linked by a covalent bond such as a phosphodiester bond, a thioester bond, a phosphorothioate bond or any of various other bonds known in the art as useful and effective for linking nucleotides. Such nucleic acid molecules can be linear, circular or supercoiled, single-stranded or double-stranded, D- or L-sterioisomers, and can be, for example, DNA or RNA, or a DNA/RNA hybrid. Nucleic acid molecules of the invention encompass those with modified linkages or containing one or more nucleoside analogs including non-naturally occurring RNA and DNA analogs; such nucleic acid molecules can exhibit resistance to enzymatic or chemical degradation.

The invention also provides vectors which contain a nucleic acid molecule encoding a SF-1-like variant or active fragment thereof having an amino acid sequence lacking at least one naturally occurring cysteine residue within the ligand-binding domain of the receptor, where the SF-1-like receptor variant or active fragment is properly folded upon expression. Such vectors, which can be cloning vectors or expression vectors, provide a means to transfer an exogenous nucleic acid molecule into a prokaryotic or eukaryotic host cell. Contemplated vectors include vectors derived from bacteria or a combination of bacterial and viral sequences, such as plasmids and cosmids, and those derived from a virus, such as a bacteriophage, baculovirus a retrovirus. The vectors of the invention can advantageously be used to clone or express a SF-1 receptor variant, LRH-1 receptor variant, or other receptor variant or active fragment of the invention. Various vectors and methods for introducing such vectors into a host cell are described, for example, in Ausubel et al., supra, 2000.

In addition to a nucleic acid molecule encoding a SF-1-like receptor variant or active fragment thereof, a vector of the invention also can contain, if desired, one or more of the following non-limiting elements: an oligonucleotide encoding, for example, a transcription or translation regulatory element or a termination codon; one or more selectable marker genes, such as an ampicillin, kanamycin, tetracycline, neomycin, hygromycin or zeomycin resistance gene, which is useful for selecting transformants or stable transfectants; one or more enhancer or promoter sequences, which can be obtained, for example, from a bacterial, viral or mammalian gene; transcription termination and RNA processing signals, which are obtained from a gene or a virus such as SV40; an origin of replication such as an SV40, polyoma or E. coli origin of replication; versatile multiple cloning sites; and one or more RNA promoters such as a T7 or SP6 promoter, which allows for in vitro transcription of sense and antisense RNA.

In one embodiment, a vector of the invention is an expression vector. Expression vectors are well known in the art and provide a means to transfer and express an exogenous nucleic acid molecule in a host cell. Contemplated expression vectors include vectors that provide for expression in a host cell such as a bacterial cell, yeast cell, insect cell, frog cell, mammalian cell or other animal cell. Such expression vectors include regulatory elements specifically required for expression of the DNA in a cell, the elements being located relative to the nucleic acid molecule encoding the SF-1-like receptor variant or active fragment so as to permit expression thereof. The regulatory elements can be chosen to provide constitutive expression or, if desired, inducible or cell type-specific expression. Regulatory elements required for expression include transcription and translation start sites and termination sites and are well known in the art. Such sites permit binding, for example, of RNA polymerase and ribosome subunits. A bacterial expression vector can include, for example, an RNA transcription promoter such as the lac promoter, a Shine-Delgarno sequence and an initiator AUG codon in the proper frame to allow translation of an amino acid sequence. The expression vector pBH (Hoffman-La Roche) is one of many expression vectors suitable for high level expression in bacteria and is specifically encompassed by the invention.

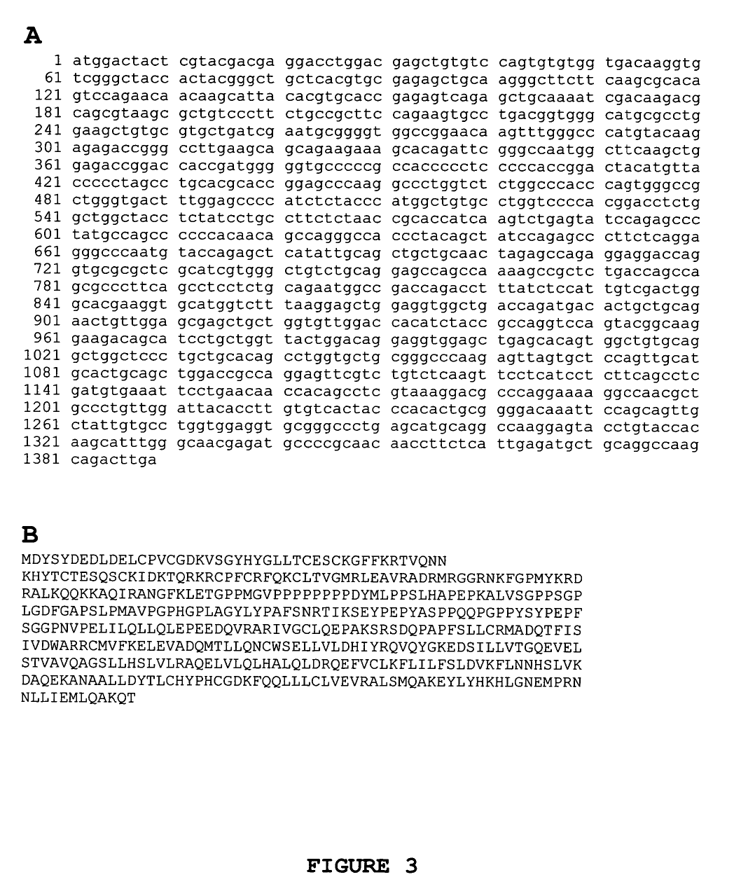

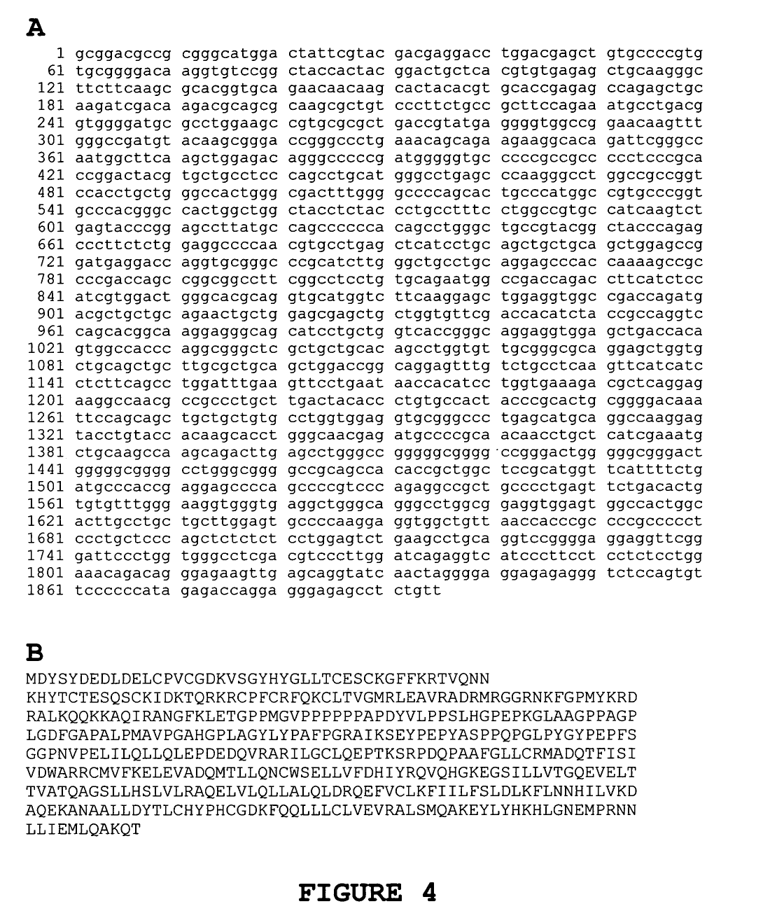

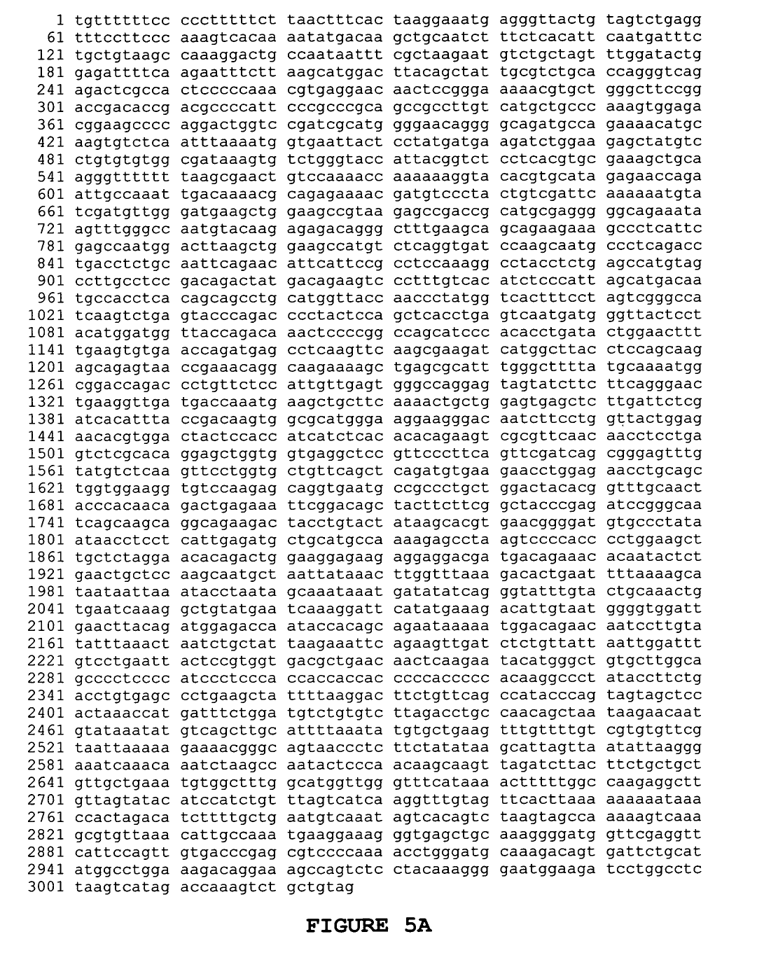

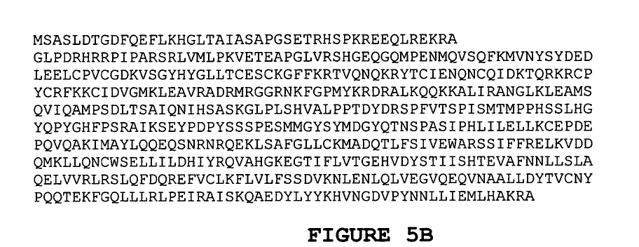

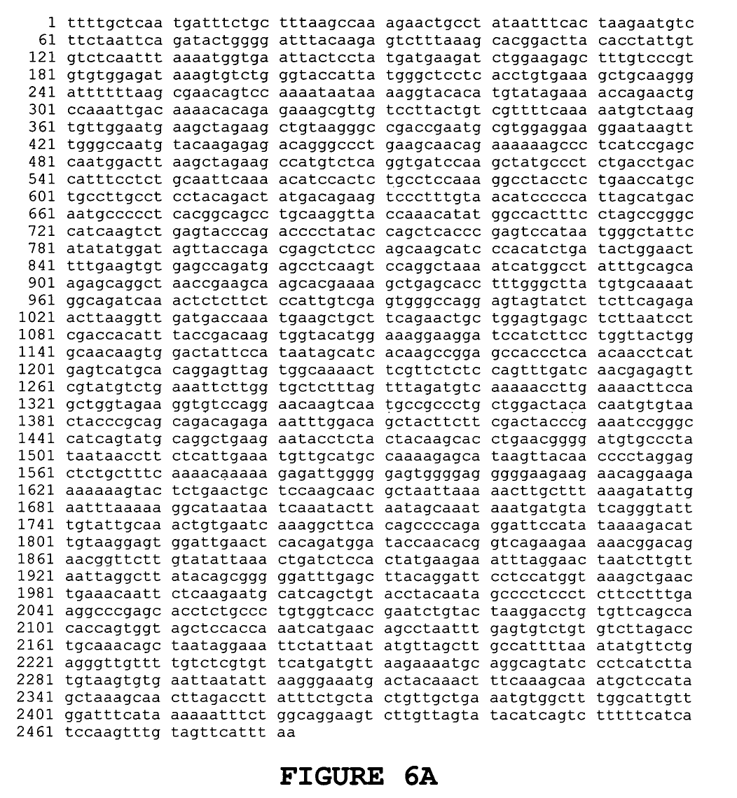

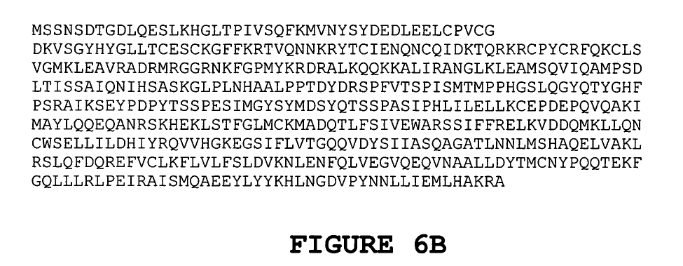

Any of a variety of routine techniques can be used to prepare a nucleic acid molecule encoding a receptor variant or active fragment of the invention based on the known sequence of wild type receptor. Wild type SF-1 and LRH-1 sequences are shown in FIGS. 3 through 6 and described hereinabove and the sequences of additional SF-1-like receptors and other monomeric nuclear receptors are well known in the art or can be obtained by routine methods.

A variety of routine molecular methods can be used to prepare a nucleic acid molecule encoding a SF-1-like receptor variant or active fragment of the invention. As a non-limiting example, oligonucleotide-directed mutagenesis can be useful in preparing the compositions of the invention. Oligonucleotide-directed mutagenesis utilizes an oligonucleotide primer encoding the desired deletion or substitution to prime DNA synthesis on a single-stranded uracil-containing template. After polymerization, for example, with T4 polymerase and addition of T4 DNA ligase, wild type bacteria are transformed, and DNA encoding the variant receptor isolated (see, for example, Smith, Ann. Rev. Genet. 19:423-463 (1985); Ausubel et al., Current Protocols in Molecular Biology Chapter 8 John Wiley & Sons, Inc. (2000)).

A nucleic acid molecule encoding a SF-1-like receptor variant such as a SF-1 or LRH-1 variant, or another monomeric nuclear receptor variant or active fragment of the invention also can be conveniently generated from a wild type sequence using the polymerase chain reaction (PCR). A synthetic oligonucleotide can be designed, for example, to incorporate a point mutation at one end of an amplified fragment. Following PCR amplification, the amplified fragment can be made blunt-ended using Klenow fragment, and then ligated and subcloned into a convenient vector for sequence analysis. Alternatively, where convenient restriction sites flank the region encoding the cysteine residue to be mutated, two oligonucleotides encompassing the mutation are annealed and extended with mutually primed synthesis. In a second PCR step, the desired fragment is amplified using outside primers, digested with the appropriate restriction enzymes and subcloned into the corresponding segment of wild type receptor. The resulting plasmid contains a fragment identical to the original wild type receptor sequence, except for the point mutation introduced by the pair of overlapping primers. These and related PCR procedures are routine and well known in the art of molecular biology. See, for example, Ausubel, supra, 2000.

A nucleic acid molecule encoding a SF-1-like receptor variant such as a SF-1 or LRH-1 receptor variant or another monomeric nuclear receptor variant or active fragment of the invention also can be assembled from mutually priming long oligonucleotides. Such a strategy is particularly useful for generating nucleic acids up to about 400 bp in length. The appropriate oligonucleotides are designed, including at least one oligonucleotide encoding the desired deletion or substitution of a naturally occurring cysteine, and typically have a length of more than 100 nucleotides. It is understood that due to degeneracy of the genetic code, codons that do not occur in the native sequence can be present in the synthetic sequence encoding the receptor variant. The two oligonucleotides are designed with complementary sequence at their 3′ end to form a short duplex segment that allows priming by the polymerase. Two or more of such pairs of oligonucleotides can be designed, if desired, with restriction endonuclease sites at the end to join adjacent fragments and for subcloning into a desired vector (Ausubel et al., supra, 2000). The skilled person understands that these and other procedures routine in the art of recombinant DNA technology can be used to prepare the nucleic acid molecules of the invention.

The invention also provides a host cell containing an expression vector that includes a nucleic acid molecule encoding a SF-1-like receptor variant or an active fragment thereof, where the SF-1-like receptor variant or active fragment is properly folded upon expression. Such a host cell can be used to express the encoded receptor variant or active fragment thereof; recombinant SF-1-like receptor variant can be substantially purified from the host cells using well known biochemical procedures (see, for example, Example I and Ausubel, supra, 2000). As disclosed herein in Example V, a SF-1-like receptor variant or active fragment of the invention is properly folded upon expression and can have one or more biological activities of the analogous wild type receptor.

Non-limiting examples of host cells useful in the invention include bacterial, yeast and frog cells, and mammalian and other animal cells. Various bacterial cells suitable for high level expression of exogenous proteins are well known in the art and include, without limitation, E. coli strains such as BL21(DE3) pLysS (Novagen, Inc.; Madison, Wis.). Various mammalian cells useful as host cells encompass, yet are not limited to, murine NIH/3T3 cells, Chinese hamster ovary (CHO) cells, COS cells and HeLa cells. Methods for introducing a vector into a host cell include electroporation, microinjection, calcium phosphate or DEAE-dextran transfection, lipofection and other methods well known in the art.

Further provided by the invention is a method of preparing an isolated properly folded SF-1-like receptor variant, or active fragment thereof, by expressing in a host cell an expression vector containing a nucleic acid molecule encoding a SF-1-like receptor variant or active fragment thereof having an amino acid sequence lacking at least one naturally occurring cysteine residue within the ligand-binding domain of the receptor, and isolating said SF-1-like receptor variant, or active fragment thereof, from the host cell. In one embodiment, the host cell is a bacterial host cell such as an E. coli BL21(DE3) pLysS cell. In another embodiment, the SF-1-like receptor or active fragment thereof contains a heterologous epitope tag, which can facilitate purification. Such an epitope tag can be, without limitation, a 6-histidine-tag, or any of the epitope tags described hereinabove or known in the art. Methods for purification of the resultant hormone receptors are described herein in Example I and also are well known in the art as described, for example, in Deutscher, Methods in Enzymology, Vol. 182, “Guide to Protein Purification,” San Diego: Academic Press, Inc. (1990).

The following examples are intended to illustrate but not limit the present invention.

### EXAMPLE I

Native SF-1 Ligand-Binding Domain Forms Dimers at High Concentrations

This examples describes purification and characterization of the AF1/LBD domain of SF-1.

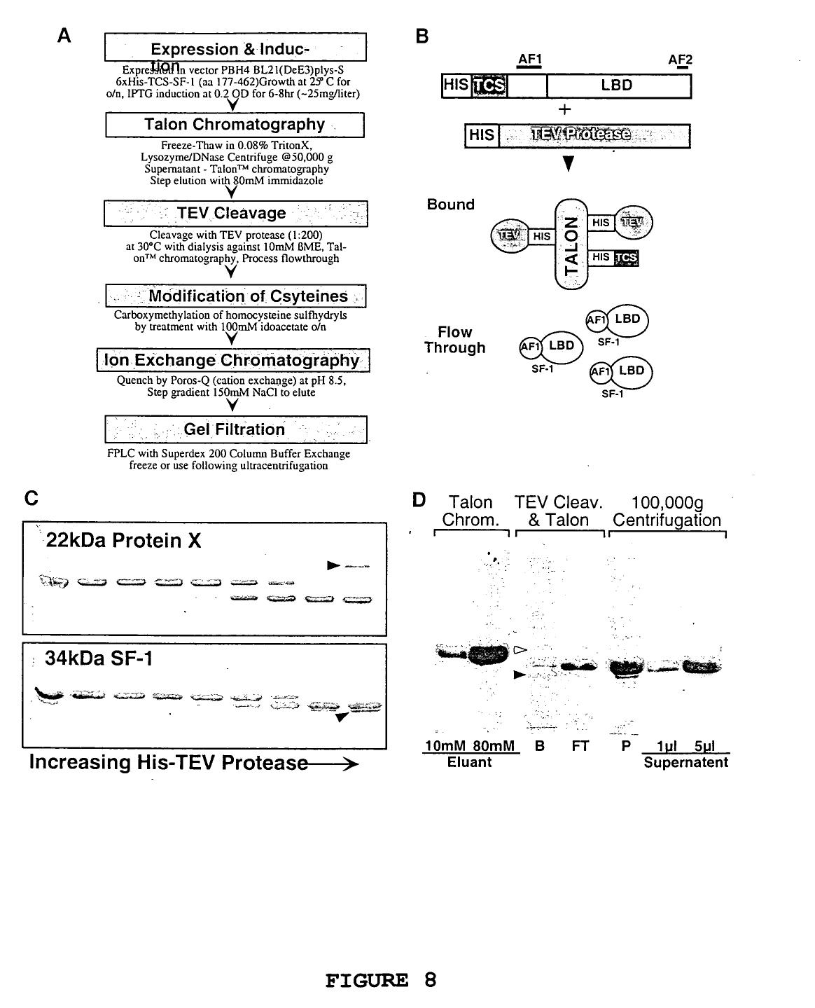

A SF-1 fragment encoding activation domain 1 and the complete ligand-binding domain (residues 177-462) was expressed in bacteria and purified using the scheme shown in FIG. 8A. The purification procedure included three chromatography steps: TALON™, ion exchange, and gel filtration chromatography as described further below.

The SF-1 protein construct encoding residues 177-462 was expressed using vector pBH in E. coli BL21(DE3) pLysS (Novagen, Inc.). Cultures were grown to 0.3 O.D. at 37° C. and subsequently transferred to room temperature, where the cultures were induced with 0.2 mM ITPG. After growth at 30° C. for five hours, cultures were harvested and frozen at −70° C.

Bacterial pellets were lysed in lysis buffer (50 mM TRIS pH 8.0, 100 mM NaCl, 5% glycerol) containing protease inhibitors. After addition of lysozyme and lysis by three cycles of freeze-thawing in liquid nitrogen, cell lysates were treated with DNase A to reduce viscosity. The concentration of KCl was then adjusted to 0.3 M, and lysates were centrifuged at 15,000 rpm in a GS 34 Sorvall rotor for 30 minutes at 4° C. The soluble fraction of His-tagged SF-1 (about 80% of total protein expressed) was purified by mixing lysates with TALON™ beads (BD Biosciences Clontech; Palo Alto, Calif.) inside a Biorad ReadyPack column (Biorad Laboratories; Hercules, Calif.). The columns were incubated with rotation for 30 minutes at 4° C. and subsequently washed with lysis buffer supplemented by 10 mM imidazole, 2 mM ATP, and 10 mM MgCl₂. His-tagged protein was eluted with an imidazole gradient (80 mM imidazole), and was typically more than 90% pure.

The 6XHIS-tag was removed from SF-1(177-462) by site-specific cleavage using the tobacco etch viral protease (TEV). The TEV recognition and cleavage site is ENLYFQ/G(TCS) (SEQ ID NO: 22) and was engineered just 5′ to the BamHI cloning site and downstream of the linker and a 6-histidine tag (see FIG. 8B). His-tagged SF-1 protein was treated with TEV protease at a ratio of 1:200 SF-1:TEV at 30° C. for 2 hours in a dialysis cassette in TEV cleavage buffer (200 mM NaCl, 100 mM TRIS pH 7.5, 20 mM β-mercaptoethanol). As shown in FIG. 8C, lower panel, incubation of the HIS-tagged SF-1 protein with increasing amounts of protease resulted in cleavage of the HIS-tag from the SF-1 fusion protein.

Dialyzed material was reapplied to TALON™, and the bound and flow through fractions analyzed. The 20 kDa TEV protease was observed as a faint band running slightly below cleaved SF-1 protein (arrow). SDS-PAGE analysis of the bound (B) and flow through (FT) fractions showed retention of the TEV protease (FIG. 8D, middle panel, black arrow) and the uncleaved HIS-TEV-SF-1 protein (FIG. 8D, middle panel, open arrow) as well as some non-specific retention of cleaved SF-1. Prior to subsequent chromatography, the flow through fraction (cleaved SF-1 protein) was concentrated and centrifuged at 100,000 g for 30 minutes to remove some minor contaminants and large SF-1(177-462) aggregates (FIG. 8D, middle panel, pellet “P”).

To reduce dimerization due to intermolecular sulfhydryl bridge formation, surface cysteines were covalently modified. The SF-1 fraction was treated overnight with 20 mM iodoacetate in 50 mM Tris pH 8.5; 50 mM NaCl; and 5 mM EDTA. Following this reaction, the SF-1(177-462) protein was subject to cation-exchange chromatography to remove minor contaminants present after the TEV cleavage and centrifugation steps and to quench the iodoacetylation reaction. SF-1(177-462) was eluted in a 150 mM NaCl step after a shallow gradient of 0-80 mM NaCl was used to separate contaminating protein from SF-1(177-462). FIG. 9, left panel, shows the A260 nM profile obtained after the cation-exchange (POROS-Q®) chromatography. Two contaminating peaks flanked the major SF-1(177-462) protein peak.

SF-1 dimers formed during the purification process were separated from the monomeric species with two back-to-back Superdex 200 gel filtrations. The results of the final Superdex 200 gel filtrations are shown in FIG. 9, right panel. The A260 nM profile shows both a minor dimeric and major monomeric SF-1(177-462) species. Native polyacrylamide gel electrophoresis confirmed the presence of dimers in the first peak (#1), and the presence of a single monomeric in the second, lower molecular weight peak (#2). These results indicate that, despite the presence of large amounts of monomeric protein, SF-1 monomer is in equilibrium with higher aggregate forms.

The yield of protein obtained with the purification procedure was greater than 50%. From 1 liter of transformed bacteria starting material, about 15 mg purified SF-1(177-462) were obtained.

### EXAMPLE II

Reduction of SF-1(177-462) Dimers by Covalent Modification of Cysteine Residues

This example demonstrates that covalent modification of cysteine residues can diminish SF-1(177-462) dimer formation.

Electrophoresis on native polyacrylamide gels indicated that SF-1(177-462) preparations contained significant amounts of dimer, trimer or larger molecular weight complexes (see FIG. 9, right panel).

Mass spectroscopy of aged SF-1(177-462) protein preparations on a Voyager DE (MALDI-TOF) instrument confirmed the presence of SF-1(177-462) dimers, as shown in FIG. 10, top panel. To determine if the amount of aggregated material can be altered by covalent modification of cysteine residues, the SF-1(177-462) preparation was treated with 20 mM iodoacetate overnight, thereby adding a carboxymethyl group to the reactive moieties that can form disulfhydryl bridges when unmodified. As shown by mass spectroscopy of iodoacetylated SF-1(177-462), the amount of dimer was reduced by about 6-fold following iodoacetylation (see FIG. 10, lower panel). These results indicate that reduction of SF-1(177-462) preparations by carboxymethylation or covalent modification of cysteine residues can be used to diminish formation of SF-1 aggregates and increase the stability of the monomeric form.

### EXAMPLE III

### Identification of Potentially Solvated Cysteines in the Murine SF-1 Ligand-Binding Domain

Potentially solvated cysteines within the SF-1 ligand-binding domain were identified by reactivity with iodoacetamide essentially as follows. Briefly, wild type SF-1 in storage buffer was diluted 1:10 in 50 mM Tris pH 8.5, 50 mM NaCl and 5 mM EDTA before iodoacetate was added to a final concentration of 20 mM and the mixture allowed to react. Following iodoacetylation, protein was treated with trypsin; mass spectrometry of the resulting tryptic fragments identified peptide sequences containing the most highly reactive cysteines. These peptides were compared to the known murine SF-1 sequence to assign cysteines 267, 302 and 423 as the most highly reactive cysteines present in SF-1.

Three dimensional modeling of the murine SF-1 ligand-binding domain was performed using the WHAT IF program (Vriend, J. Mol. Graphics 8:52-56 (1990)) based on the structure of human RXRα, where sequences for murine SF-1 were aligned against the sequence of the PDB entry of the crystal structure of the liganded LBD, chain A (Egea et al., EMBO J. 19:2592-2601 (2000)). Models were constructed as described previously in Vriend, supra, 1990. From the three dimensional model of the murine SF-1 ligand-binding domain, amino acids 408 and 413 were identified as additional potentially solvated cysteine residues.

In sum, these results identify cysteines C267, C302, C408, C413 and C423 as potentially solvated and available for intermolecular disulfide bond formation..

### EXAMPLE IV

### Preparation OF SF-1 Ligand-Binding Domain Variants Containing Serine for Cysteine Substitutions

This example describes preparation of SF-1(219-462) variants containing two,. three or four cysteine to serine substitutions within the ligand-binding domain.

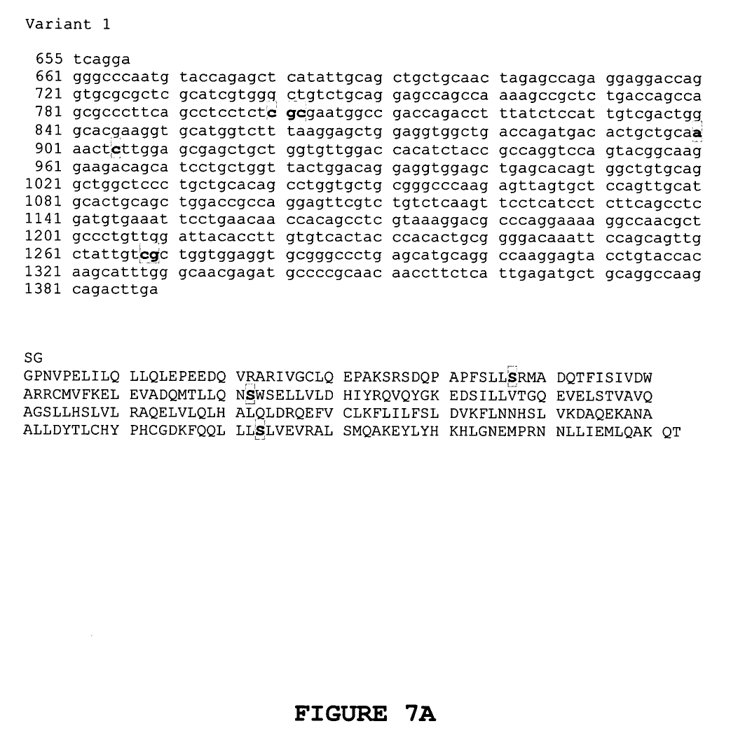

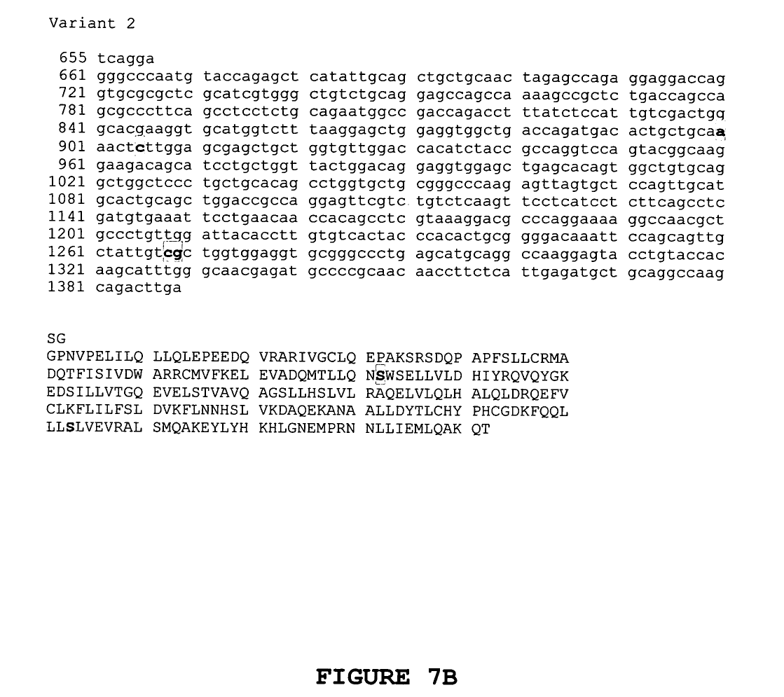

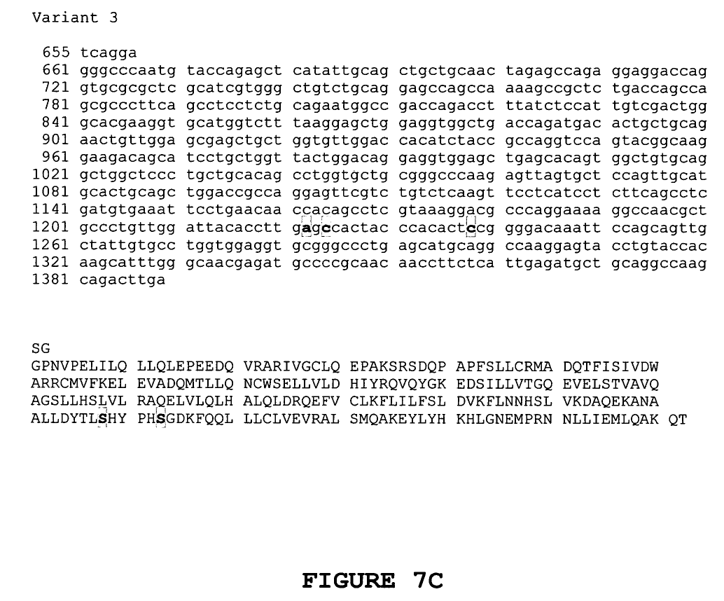

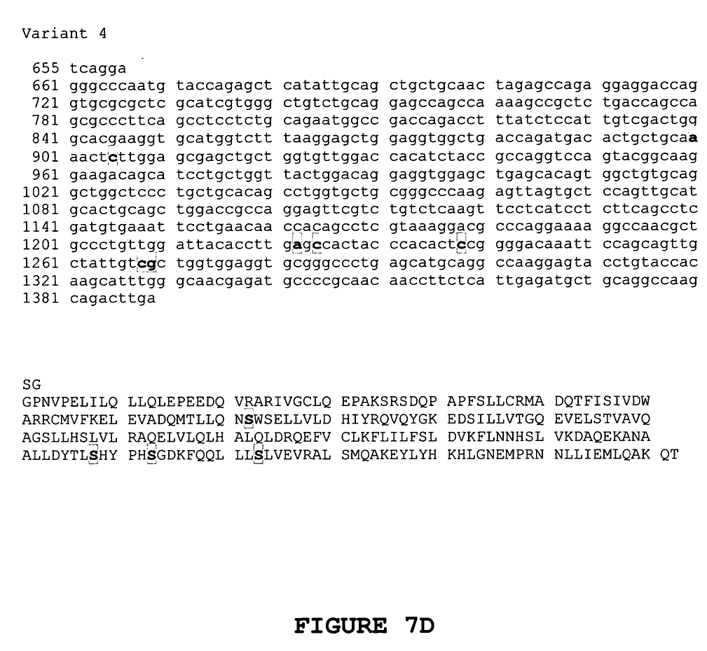

Constructs were designed to contain substitutions at two or more cysteines based on the identification of potentially solvated cysteines described above. As for the wild type SF-1(219-462) construct, each variant was cloned into BamHI and XhoI sites and contained a 6-histidine linker separated from residues 219 to 462 of SF-1 by a TEV protease site. Each of the SF-1 variants was designed to have a Gly-Ser amino-terminal sequence. The nucleic acid sequences of the SF-1 coding portions of Variant 1 (SEQ ID NO: 14), Variant 2 (SEQ ID NO: 16), Variant 3 (SEQ ID NO: 18) and Variant 4 (SEQ ID NO: 20) are shown in FIG. 7.

Constructs were prepared by Kunkel mutagenesis according to the method described in Kunkel et al., Meth. Enzymol. 154: 367 (1987), using the primers shown in Table 1.

TABLE 1

| Primer | Mutation | Sequence/SEQ ID NO: | |
|---|---|---|---|
| A, reverse | C267S | 5′-TCGGCCATTCGCGAGAGGAGGCTG-3′† | (SEQ ID NO:28) |
| B, reverse | C302S | 5′-CTCGCTCCAAGAGTTTTGCAGCAGTG-3′ | (SEQ ID NO:29) |
| C, reverse | C423S | 5′-CACCTCCACCAGCGACAATAGCAAC-3′ | (SEQ ID NO:30) |
| D, forward | C408S, | 5′-TACACCTTGAGCCACTACCCACACTCCG | (SEQ ID NO:31) |
| | C413S | GGGACAAATTCC-3′ | |
| BamHI, | ---- | 5′-CTATCCAGAGGGATCCTCAGGAGG-3′* | (SEQ ID NO:32) |
| forward | | | |
| XhoI, reverse | ---- | 5′-AGGAGTCTTCTCGAGGCAGTGGCA-3′ | (SEQ ID NO:33) |

†Nucleotide substitutions underlined and in bold
*Restriction sites indicated in bold

To create Variant 1, Primer A (SEQ ID NO: 28), Primer B (SEQ ID NO: 29), and Primer C (SEQ ID NO: 30) were used to produce serine substitutions at C267, C302 and C423, generating the mutations in Variant 1. Primer D (SEQ ID NO: 31) was used to introduce serine substitutions at cysteines C413 and C423, producing the mutations in Variant 3. To create Variant 4, Variant 3 was used as the template for mutagenesis with Primers B (SEQ ID NO: 29) and C (SEQ ID NO: 30), thereby adding serine substitutions at C302S and C423S.

Using the three different variant templates produced above, the region spanning murine SF-1 amino acids 219 to 462 was amplified using forward BamHI primer SEQ ID NO: 32 and reverse XhoI primer SEQ ID NO: 33. The amplified segments containing newly created 5′ BamH1 and 3′ XhoI sites were ligated into pBH4, which is a modification of the vector pET19b that contains a 6xHis tag and a cleavage site for the tobacco etch virus (TEV) protease (Hillier et al., Science 284: 812-815 (1999).).

The SF-1 variants were purified as described above in Example I with omission of iodoacetylation steps.

The following yields were obtained.

TABLE 2
Purification of wild type SF-1 and receptor variants

| Construct | % Yield | % Purity |
|---|---|---|
| Wild type SF-1 | about 50% | 99% (aggregated) |
| Variant 1 | about 50% | 99% |
| Variant 3 | about 50% | 99% |
| Variant 4 | Not available* | Not available* |

*Expressed protein was insoluble

### EXAMPLE V

Characterization of SF-1(219-462) Variants Containing Serine for Cysteine Substitutions

This example demonstrates that cysteine to serine substitutions within the SF-1 receptor ligand-binding domain can be used to produce properly folded receptor variants that exhibit increased monomer stability at high concentration as compared to the stability of the wild type SF-1 AF1/ligand-binding domain.

Thermal denaturation of SF-1(219-462) variant proteins was measured by monitoring circular dichroism (CD) spectra at 222 nm using a Jasco model J-720 spectrophotometer with Peltier temperature controller and 1 mm path cuvettes. Purified proteins (10 μM) were equilibrated in CD buffer (20 mM TRIS pH 8.0, 150 mM NaCl, 10% glycerol, 3 mM CHAPS, 2 mM TCEP) and centrifuged prior to all measurements. Profiles were determined by increasing the temperature from 20° C. to 90° C. with a gradient of 30° C. per hour (an acquisition time of 4 seconds) with a data pitch interval of 0.2° C. Midpoints of the thermal transitions were estimated using the first derivations of the thermal denaturation curves.

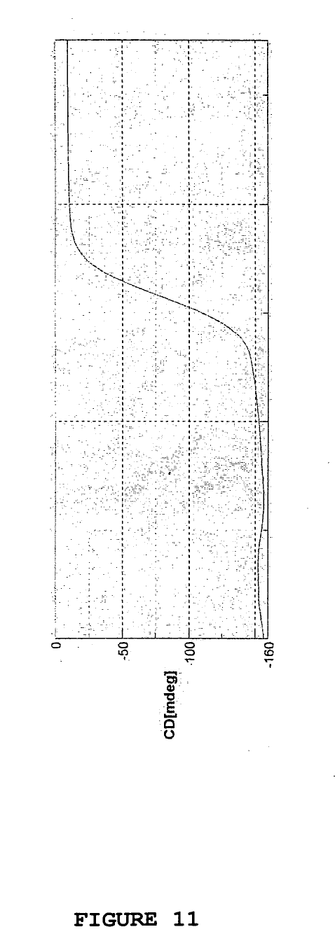

As shown in FIG. 11, SF-1 Variant 3 displayed a single melting transition point, indicative of a properly folded protein. The melting point of the protein was approximately 48° C. These results demonstrate that substitution of cysteines within the ligand-binding domain of SF-1 does not greatly perturb the structure of the ligand-binding domain and that this strategy can be used to prepare properly folded SF-1 and other receptor variants.

Dynamic light scattering analysis was performed to determine whether SF-1 variant protein was monodispersed (monomeric) and also to measure the radius of the protein. Samples were dialyzed and assayed at 50-120 μM in the following buffer: 20 mM HEPES, pH 7.4; 100 mM NaC; 1 mM EDTA; 1 mM CHAPS; and 10 mM DTT as described in Brown (Ed.), Dynamic Light Scattering: The Method and Some Applications Oxford: Clarendon Press (1993).

As shown in FIG. 12B, preparations of SF-1 Variant 1 (C267S/C302S/C423S) were about 80% monomeric. Analysis of SF-1 Variant 3 (C408S/C413S; SEQ ID NO: 19) revealed that this mutant protein was more than 91.4% monomeric, with a stokes radius of 2.34 nm and a size of 25 kDa (see FIG. 12C). Unlike SF-1 variants 1 and 3, the fraction of wild type SF-1 (219-462) was too low for accurate quantitation, as shown in FIG. 12A. In contrast to wild type SF-1(219-462), relatively little aggregated protein was observed for either variant.

These results indicate that SF-1 Variants 1 and 3 display a single stable monomeric species in contrast to the scatter observed with wild type SF-1 ligand-binding domain.

SF-1 Variants 1 and 3 were subject to nuclear magnetic resonance (NMR) analysis. As shown in FIG. 13, Variant 3 showed less aggregation, greater solubility (100 μM), and greater intensity and definition of peaks than SF-1 Variant 1. Both cysteine variants also displayed significantly less aggregation than wild type protein. These data indicate that SF-1 Variants 1 and 3 have greater overall stability of monomeric configuration than the same portion of unmodified (wild type) SF-1.

Fluorescent peptides were used in equilibrium binding studies with SF-1 Variant 3 to determine if the variant was functional for coactivator binding. Coactivator peptides tested in the assay were selected from a set of 40 known nuclear receptor coactivators and corepressors and included all NR interaction boxes from GRIP1, SRC, PGC, NcoR, Smrt, Trap, Prip and Rip140. Control coactivator peptides harbored mutations of Leucine to Alanine in positions 4 and 5 of the LXXLL motif (LXXAA). Binding was performed essentially as described in Geistlinger and Guy, J. Am. Chem. Soc. 123:1525-1526 (2001), in the following binding buffer: 20 mM Tris HCl pH 8.0, 100 mM NaCl, 10% glycerol, 1 mM DTT, 0.01% NP-40 and 1 mM EDTA. Binding data with different peptides and their controls were obtained; the results with GRIP1 and SRC peptides and SF-1 Variant 3 are illustrated.

As shown in the FIG. 14, SF-1 Variant 3 bound specifically to GRIP peptides; this binding was not saturable up to 100 μM of protein. As expected, SRC coactivator peptides did not display specific binding to SF-1 Variant 3. In contrast, wild type SF-1(219-462) bound neither GRIP nor SRC coactivator peptides, consistent with the fact that the wild type ligand binding domain was present as inactive dimers or other aggregates.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

We claim:

1. A properly folded steroidogenic factor-1 (SF-1)-like receptor variant or active fragment thereof, comprising an amino acid sequence encoding a SF-1-like receptor or an active fragment thereof, said amino acid sequence lacking at least one naturally occurring cysteine residue within the ligand-binding domain of said receptor.

2. The receptor variant or active fragment of claim 1, which exhibits increased monomer stability as compared to analogous receptor retaining all cysteine residues lacking in said variant.

3. The receptor variant or active fragment of claim 1, wherein said cysteine residue is substituted with a non-cysteine residue.

4. The receptor variant or active fragment of claim 1, wherein said SF-1-like receptor is SF-1.

5. The receptor variant or active fragment of claim 1, wherein said SF-1-like receptor is LRH-1/FTF/SF-1β.

6. The receptor variant of claim 1, 3, 4 or 5, which is an active fragment comprising a ligand-binding domain.

7. The receptor variant fragment of claim 6, which does not contain additional sequence derived from said receptor.

8. The receptor variant of claim 1, 3, 4 or 5, which is an active fragment comprising a ligand-binding domain and activation function 1 (AF1).

9. The receptor variant fragment of claim 8, which does not contain additional sequence derived from said receptor

10. The receptor variant of claim 1, 3, 4 or 5, said amino acid sequence lacking at least two naturally occurring cysteine residues within the ligand-binding domain of said receptor.

11. The receptor variant of claim 10, wherein said at least two naturally occurring cysteine residues are substituted with non-cysteine residues.

12. The receptor variant of claim 1, 3, 4 or 5, said amino acid sequence lacking at least three naturally occurring cysteine residues within the ligand-binding domain of said receptor.

13. The receptor variant of claim 12, wherein said at least three naturally occurring cysteine residues are substituted with non-cysteine residues.

14. The receptor variant of claim 3, 11 or 13, wherein said cysteine residue or residues each is independently substituted with an amino acid selected from the group consisting of serine, threonine, alanine, valine, glycine, leucine, isoleucine, tyrosine, phenylalanine, tryptophan, methionine and histidine.

15. The receptor variant of claim 14, wherein said cysteine residue or residues each is independently substituted with serine or threonine.

16. The receptor variant of claim 1 or 3, said variant comprising amino acid substitutions at cysteine residues corresponding to C267, C302 and C423 of murine SF-1.

17. The SF-1 receptor variant of claim 4, said variant comprising amino acid substitutions at cysteine residues corresponding to C267, C302 and C423 of murine SF-1.

18. The SF-1 receptor variant of claim 17, said variant comprising serine substitutions at cysteine residues corresponding to C267, C302 and C423 of murine SF-1.

19. The SF-1 receptor variant of claim 17 or 18, wherein said variant is a human SF-1 receptor variant.

20. The receptor variant of claim 1 or 3, said variant comprising amino acid substitutions at cysteine residues corresponding to C302 and C423 of murine SF-1.

21. The SF-1 receptor variant of claim 4, said variant comprising amino acid substitutions at cysteine residues corresponding to C302 and C423 of murine SF-1.

22. The SF-1 receptor variant of claim 21, said variant comprising serine substitutions at cysteine residues corresponding to C302 and C423 of murine SF-1.

23. The SF-1 receptor variant of claim 21 or 22, wherein said variant is a human SF-1 receptor variant.

24. The receptor variant of claim 1 or 3, said variant comprising amino acid substitutions at cysteine residues corresponding to C408 and C413 of murine SF-1.

25. The SF-1 receptor variant of claim 4, said variant comprising amino acid substitutions at cysteine residues corresponding to C408 and C413 of murine SF-1.

26. The SF-1 receptor variant of claim 25, said variant comprising serine substitutions at cysteine residues corresponding to C408 and C413 of murine SF-1.

27. The SF-1 receptor variant of claim 25 or 26, wherein said variant is a human SF-1 receptor variant.

28. A properly folded SF-1 receptor variant or active fragment thereof, comprising an amino acid sequence encoding a SF-1 receptor or an active fragment thereof, said amino acid sequence lacking at least one naturally occurring cysteine residue selected from the group consisting of
a cysteine residue corresponding to C408 of murine SF-1 and
a cysteine residue corresponding to C413 of murine SF-1.

29. The SF-1 receptor variant or active fragment of claim 28, which exhibits increased monomer stability as compared to analogous receptor retaining all cysteine residues lacking in said variant.

30. The SF-1 receptor variant or active fragment of claim 28, wherein said at least one cysteine residue is substituted with a non-cysteine residue.

31. The SF-1 receptor variant or active fragment of claim 28, comprising an amino acid sequence lacking naturally occurring cysteine residues corresponding to C408 and C413 of murine SF-1.

32. The SF-1 receptor variant or active fragment of claim 31, said variant comprising amino acid substitutions at cysteine residues corresponding to C408 and C413 of murine SF-1.

33. The SF-1 receptor variant or active fragment of claim 32, wherein each of said cysteine residues is independently substituted with an amino acid selected from the group consisting of serine, threonine, alanine and valine.

34. The SF-1 receptor variant or active fragment of claim 33, wherein each of said cysteine residues is independently substituted with an amino acid selected from the group consisting of serine and threonine.

35. The SF-1 receptor variant or active fragment of claim 34, wherein each of said cysteine residues is substituted with serine.

36. The SF-1 receptor variant or active fragment of claim 28, 33, 34 or 35, which is an active fragment comprising a ligand-binding domain.

37. The SF-1 active fragment of claim 36, which does not contain additional SF-1 receptor sequence.

38. The SF-1 receptor variant or active fragment of claim 28, which is a human SF-1 receptor variant.

39. The SF-1 receptor variant or active fragment of claim 28, which is a murine SF-1 receptor variant.

40. The murine SF-1 receptor variant or active fragment of claim 39, comprising the amino acid sequence SEQ ID NO: 19.

41. A properly folded SF-1 receptor variant or active fragment thereof, comprising an amino acid sequence encoding a SF-1 receptor or an active fragment thereof, said amino acid sequence lacking at least one naturally occurring cysteine residue selected from the group consisting of
a cysteine residue corresponding to C267 of murine SF-1;
a cysteine residue corresponding to C302 of murine SF-1; and
a cysteine residue corresponding to C423 of murine SF-1.

42. The SF-1 receptor variant or active fragment of claim 41, which exhibits increased monomer stability as compared to analogous receptor retaining all cysteine residues lacking in said variant.

43. The SF-1 receptor variant or active fragment of claim 41, wherein at least one naturally occurring cysteine residue is substituted with a non-cysteine residue.

44. The SF-1 receptor variant or active fragment of claim 41, comprising an amino acid sequence lacking naturally occurring cysteine residues corresponding to C267 and C302 of murine SF-1.

45. The SF-1 receptor variant or active fragment of claim 44, said variant comprising amino acid substitutions at cysteine residues corresponding to C267 and C302 of murine SF-1.

46. The SF-1 receptor variant or active fragment of claim 41, comprising an amino acid sequence lacking naturally occurring cysteine residues corresponding to C267 and C423 of murine SF-1.

47. The SF-1 receptor variant or active fragment of claim 46, said variant comprising amino acid substitutions at cysteine residues corresponding to C267 and C423 of murine SF-1.

48. The SF-1 receptor variant or active fragment of claim 41, comprising an amino acid sequence lacking naturally occurring cysteine residues corresponding to C302 and C423 of murine SF-1.

49. The SF-1 receptor variant or active fragment of claim 48, said variant comprising amino acid substitutions at cysteine residues corresponding to C302 and C423 of murine SF-1.

50. The SF-1 receptor variant or active fragment of claim 41, comprising an amino acid sequence lacking naturally occurring cysteine residues corresponding to C267, C302 and C423 of murine SF-1.

51. The SF-1 receptor variant or active fragment of claim 50, said variant comprising amino acid substitutions at cysteine residues corresponding to C267, C302 and C423 of murine SF-1.

52. The SF-1 receptor variant or active fragment of claim 45, 47, 49 or 51, wherein each of said cysteine residues is independently substituted with an amino acid selected from the group consisting of serine, threonine, alanine and valine.

53. The SF-1 receptor variant or active fragment of claim 45, 47, 49 or 51, wherein each of said cysteine residues is independently substituted with an amino acid selected from the group consisting of serine and threonine.

54. The SF-1 receptor variant or active fragment of claim 45, 47, 49 or 51, wherein each of said cysteine residues is substituted with serine.

55. The SF-1 receptor variant or active fragment of claim 41, which is an active fragment comprising a ligand-binding domain.

56. The SF-1 active fragment of claim 55, which does not contain additional SF-1 receptor sequence.

57. The SF-1 receptor variant or active fragment of claim 41, which is a human SF-1 receptor variant.

58. The SF-1 receptor variant or active fragment of claim 41, which is a murine SF-1 receptor variant.

59. The murine SF-1 receptor variant or active fragment of claim 58, comprising the amino acid sequence SEQ ID NO: 15.
